(19) United States
(12) Patent Application Publication
(10) Pub. No.: US 2006/0040884 A1
(43) Pub. Date: Feb. 23, 2006

(54) Antisense oligonucleotides directed to genes regulated by trapoxin-induced HDAC inhibition

(76) Inventors: Francis P. Buxton, Morristown, NJ (US); Dalia Cohen, Livingston, NJ (US); Denise D. Fischer, Bernardsville, NJ (US); Shaowen Wang, Edison, NJ (US)

Correspondence Address: Novartis;Corporate Intellectual Property, Building 104, One Health Plaza, East Hanover, NJ, 07936-1080, US

(21) Appl. No.: 11/191,875

(22) Filed: Jul. 28, 2005

Related U.S. Application Data
(63) Continuation of application No. 10/154,659, filed on May 23, 2002, now abandoned.
(60) Provisional application No. 60/293,090, filed on May 23, 2001.
(60) Provisional application No. 60/344,416, filed on Nov. 7, 2001.
(60) Provisional application No. 60/370,332, filed on Apr. 5, 2002.

(51) Int. Cl. A61K 48/00; C07H 21/04

(52) U.S. Cl. 514/44; 536/23.1

(57) ABSTRACT

Antisense compounds, compositions and methods are provided for modulating the expression of trapoxin A regulated genes, including but not limited to those genes induced by ectopic expression of p21^{waf1} that are disclosed herein. The compositions comprise antisense compounds, particularly antisense oligonucleotides, targeted to nucleic acids encoding these genes. Methods of using these compounds for modulation of expression of these genes and for treatment of diseases associated with these genes, as well as those associated with abnormal HDAC activity, particularly cancer or others characterized by abnormal cell proliferation, are provided. Furthermore, the invention relates to the use of RhoB as a biomarker to evaluate the efficacy of treatment of humans with abnormal HDAC activity including proliferative diseases such as cancer. Also disclosed is a method for identifying HDAC inhibitors and trapoxin analogs based on the surprising discovery that up regulation or RhoB and increased RhoB protein levels are associated with HDAC inhibition.

### FIELD OF THE INVENTION

This invention provides methods for treating or preventing conditions associated with abnormal HDAC activity in humans comprising modulating the expression of trapoxin A regulated genes. In particular, this invention relates to antisense compounds, particularly oligonucleotides, specifically hybridizable with nucleic acids encoding human trapoxin A down-regulated genes. The invention also relates to the use of trapoxin A regulated genes as a marker for HDAC inhibiton in a method to screen a library of agents for HDAC inhibitory activity.

### BACKGROUND OF THE INVENTION

Measuring the levels of many messages simultaneously in response to drug treatment or disease progression can give important insight into disease processes by revealing new functions for known genes. Furthermore, changes in transcriptional regulation can be used to reconstruct signalling pathways that are effected by the disease or drug treatment. The most cost effective method for profiling large numbers of genes is by hybridization of complex probes made from RNA to arrays of DNA on solid supports. In complex probes all transcripts are present at a rather low level. Scoring differentially expressed genes in a quantitative fashion not only allows the identification of potential new targets; it might identify marker genes for drug action to be used in later stages of drug discovery programs.

Several possible approaches exist for a systematic evaluation of genes identified as candidates for new drug targets. One of these, antisense technology, is a particularly powerful technique well suited for filtering lists of genes in this respect. Antisense technology contributes to this filter process by providing rationally-designed gene inhibitors solely from knowledge of part of the primary gene sequence. In other words, gene inhibitors can be rationally designed from an EST sequence without any additional knowledge of the target. Testing of these inhibitors in a suitable assay can reveal whether the gene contributes causally to a particular phenotype. Genes whose inhibition of expression by oligonucleotides leads to a change in phenotype, are potential tagets and may then, in a second step, be subjected to the usual investigative process culminating in validation of the gene as a new pharmaceutical target.

Changes in core histone acetylation by histone acetylases (HATs), histone deacetylases (HDAC), and nucleosome disruption by ATP-dependent remodelling complexes have been shown to play important roles in the regulation of transcription. In normal cells, acetylation of histones and nucleosome disruption is generally thought to facilitate transcription, whereas HDACs function as components of complexes that are involved in transcriptional repression.

Deregulation of histone acetylases and deacetylases were found to be associated with tumorigenicity. Truncation of p300 acetylase was found in epithelial cancers and steroid receptor cofactor SRC-3 (p/CIP/RAC3/AIB1/ACTR/TRAM-1) is overexpressed in steroid-inducible cancer. Furthermore, cyclic AMP response element-binding protein (CREB)-binding protein (CBP) is inappropriately fused to MOZ and MLL proteins in acute myeloid leukemia. HDAC1 was found associated with mutant fusion proteins of retinoic acid receptors PML-RAR-α and PLZF-RAR-α and contributed to the retinoid resistance of acute promyelocytic leukemia (APL).

Currently 17 human HDAC isoforms have been reported and these HDACs are divided into three classes based upon sequence homology. Class I HDACs 1-3 and 8 are similar to the yeast gene Rpd3, class II HDACs 4-7 are similar to yeast gene hda1, and class III HDACs Sirt1-7 are homologs of yeast Sir2. Class I HDAC complexes were found to contain co-repressors including mSin3A and mSin3A-associated proteins, silencing mediators nuclear receptor corepressor and silencing mediator for retinoid and thyroid hormone receptors, transcriptional repressors retinoblastoma protein (Rb), Rb-like proteins p107, p130, and Rb-associated proteins. Furthermore, Mad/Max, nuclear hormone receptors, nucleosome remodelling factors, methyl-binding proteins, and DNA repair machinery proteins were also found in association with class I HDACs. In addition, HDAC1 has been found to bind directly to YY1, Sp1, and to other HDAC family members. Class II HDACs do not associate with the Sin3 core complex, but have been found in complexes with proteins that are so far, unique to class II HDACs. HDAC4 phosphorylation and association with importin α were found to be important determinants of HDAC4 cellular localization. Hypo-phosphorylated HDAC4 is imported into the nucleus when bound to importin α, whereas phosphorylated HDAC4 is associated with 14-3-3 β and ε and is sequestered in the cytoplasm. Map kinase kinases, Erks1 and 2 were also found to associate with HDAC4, suggesting a possible mechanism of HDAC4 phosphorylation.

Several classes of compounds have been found to inhibit HDACs and there has been some clinical success using the HDAC inhibitor, trichostatin A as an adjunct therapy in the treatment of retinoic acid-refractory PML-RARα APL (Grignani, F. et al. 1998 Nature 39:815-818). Several other HDAC inhibitors are being studied and some are nearing the clinic (Byrd, J. C. et al. 1999 Blood 94:1401-08; Kim, Y. B et al., 1999 Oncogene 18:2461-2470; Cohen, L. A. 1999 AntiCancer Res. 19:4999-5005). Trapoxin A (TPX) is the first HDAC inhibitor that was found to covalently bind to HDAC1 and lead to its irreversible inhibition (Yoshida, M. et al., 1995 Bioessays 17:423-430).

Trapoxin A (TPX), a microbially derived cyclotetrapeptide (Itazaki et al. J. Antibiot. 43(12):1524-1532 (1990)) has been shown to bind to and potently inhibit histone deacetylase 1 (HDAC1) (Tauton et al. Science 272:408-411 (1996)). The inhibition of HDAC1 by Tpx interferes with mSin3 association and transcriptional repression. The complex of mSin3 with N-CoR and Mad is thought to repress transcription and to play a negative role in the regulation of cell proliferation. It has been suggested that histone deacetylation can contribute to repression of specific genes. In addition, HDAC inhibitors synergize with retinoic acid to stimulate hormone responsive genes and differentiation of myeloid leukemia HL-60 cells. Numerous anti-proliferative effects have also been reported for agents that inhibit histone deacetylation. These effects include induction of cell cycle arrest at G1 and G2 and in vitro differentiation of certain transformed cell lines (Yoshida and Beppu, Exp. Cell Res. 177:122-131 (1988), Itazaki et al. J. Antibiot. 12:1524-1532 (1990), Yoshida et al. J. Antibiot. 43:1101-1106 (1990), Hoshikawa et al. Agric. Biol. Chem. 55:1491-1497 (1991), Sugita et al. Cancer Res. 52:168-172 (1992), Yoshida and Sugita, J. Antibiot., 12:1524-1532, (1990), Hoshikawa et al. Exp. Cell Res. 214:189-197 (1994) or apoptosis of transformed cells (Medina et al. Cancer Res. 57(17):3697-3707 (1997).

A number of genes have been found to be transcriptionally repressed as a response to the binding HDAC-containing complexes to their promoters, including E2F-responsive gene cyclin E (Brehm, A. et al., (1998) Nature 391, 597-601), Bax (Juan, L. J. et al., (2000) J. Biol. Chem., 275 20436-20443), myocyte enhancer factor-2-dependent genes (McKinsey, T. A. et al. (2000) Nature 408, 106-111), human immunodeficiency virus type 1 long terminal repeat (Coull, J. J., et al. (2000) J. Virol. 74, 6790-6799) and transcriptional repression in response to thyroid hormone and retinoic acid receptors (Fu, M. et al. (2000) J. Biol. Chem. 275, 20853-20860). In addition, HDAC inhibition through treatment with chemical inhibitors has been found to modulate the transcription of gelsolin (Hoshikawa, Y. et al., (1994) Exp. Cell. Res. 214. 189-197), urokinase plasminogen activator (Upa), its receptor UpaR, and inhibitor PAI-1 (Reeder, J. A. et al., (1993) Teratog. Carcinog. Mutagen. 13, 75-88; Dong-Le Bourhis, X. et al. (1998) Br. J. Cancer 77, 396-403), multi-drug resistance gene 1 (Jin, S. and Scotto, K. W. (1998) Mol. Cell. Biol. 18, 4377-4384), and cyclin-dependent kinase inhibitor p21^{waf1} (Sambucetti, L. C. et al., (1999) J. Biol. Chem. 274, 34940-34947; Sowa, Y. et al (1999) Ann. N.Y. Acad. Sci. 886, 195-199; Kardassis, D. et al. (1999) J. Biol. Chem. 274, 29572-29581). These responses to chemically mediated HDAC inhibition suggest a broad role of HDACs in the regulation of gene transcription. However, the downstream effects of HDAC inhibition as a result of treatment with HDAC inhibitors are not well understood.

Previously, it was demonstrated that p21^{waf1} is induced upon treatment with HDAC inhibitors. The induction of p21^{waf1} in tumor cells resulted in cell growth arrest or apoptosis. p21^{waf1} is a cyclin-dependent kinase inhibitor that regulates the cell cycle G₁-phase checkpoint and inhibits proliferating cell nuclear antigen (PCNA)-mediated DNA replication during S-phase. p21^{waf1} is also an important downstream mediator of p53 and DNA damage that inhibits nucleotide excision repair through the binding of its C-terminus to PCNA. Furthermore, expression of p21^{waf1} in p53-wild type cells was found to inhibit mitotic control genes, a pattern that has been associated with an aging cellular phenotype.

HDAC inhibition through treatment with chemical inhibitors of HDACs including TPX, trichostatin A, and butyrate induce p21^{waf1} transcription in a p53-independent manner in a region of its promoter that contains Sp1 (Eilers, A. L. et al., (1999) J. Biol. Chem. 274 32750-32756; Sambucetti, L. C. et al., (1999) J. Biol. Chem. 274, 34940-34947; Sowa, Y. et al (1999) Ann. N.Y. Acad. Sci. 886, 195-199; Kardassis, D. et al. (1999) J. Biol. Chem. 274, 29572-29581), and Sp3 (Grignani, F. et al., (1998) Nature 39, 815-818; Xiao, H., et al. (1999) J. Cell. Biochem. 73, 291-302) sites. In addition, the p21^{waf1} promoter is acetylated in this region of the promoter within the chromatin complex. Another zinc-finger transcription factor that induces collagen gene transcription (BFCOL/ZBP-89) was also found to bind and transactivate the p21^{waf1} promoter in a GC-rich region proximal to these Sp1 sites. Furthermore, BFCOL/ZBP-89 protein complexes with p300 acetylase. Together this evidence suggests a mechanism for p21^{waf1} promoter regulation in response to changes in chromatin acetylation.

This invention describes a genomic approach to examine the expression profiles of H1299 cells that expressed p21^{waf1} either ectopically or in response to HDAC inhibition by TPX treatment. These studies identified transcripts that are differentially expressed in response to p21^{waf1} expression or HDAC inhibition by TPX treatment. The number of genes that show similar expression profiles in response to p21^{waf1} expression and HDAC inhibition by TPX treatment is limited. However, many of these genes are involved in similar processes, including the cell cycle, proliferation, and DNA replication. While most of the transcripts that are altered are of previously known function, many have not previously been associated with HDAC inhibition.

An understanding of the biological events associated with the antiproliferative effect characteristic of compounds such as trapoxin A would be useful for the development of new therapies. Particularly, in view of the present use of HDAC inhibitors in clinical trials as cancer therapies, it is of utmost importance that the consequences of HDAC inhibition are understood. For example, information regarding genes that may be upregulated or downregulated in a cell exposed to trapoxin A (i.e. in a cell in which HDACs are inhibited) could provide the basis for new therapies to treat diseases or other conditions associated with abnormal cell growth, including but not limited to, conditions associated with abnormal HDAC activity or gene expression (e.g., abnormal cell proliferation, cancer, atherosclerosis, inflammatory bowel disease, host inflammatory or immune response or psoriasis).

### SUMMARY OF THE INVENTION

In one aspect, the invention is directed to antisense compounds, particularly oligonucleotides, which are targeted to nucleic acids encoding genes that are down-regulated in vitro by trapoxin A treatment or p21^{waf1} overexpression.

Pharmaceutical and other compositions comprising the antisense compounds of the invention are also provided. Further provided are methods of modulating expression of these trapoxin A regulated genes in cells or tissues comprising contacting said cells or tissues with one or more of the antisense compounds or compositions of the invention. Further provided are methods of treating an animal, particularly a human, suspected of having or being prone to a disease or condition associated with expression of a trapoxin A down-regulated gene by administering a therapeutically or prophylactically effective amount of one or more of the antisense compounds or compositions of the present invention.

In another aspect, the invention pertains to a method for screening a compound for HDAC inhibitory activity, comprising administering said compound to a subject and assaying for RhoB mRNA levels in a biological sample from said subject wherein increased levels compared to controls indicate a compound possessing HDAC inhibitory activity.

In another aspect, the invention pertains to a method for screening a compound for HDAC inhibitory activities, comprising administering said compound to an in vitro cellular screening system and assaying for RhoB mRNA levels in said system wherein increased levels compared to controls indicate a compound possessing HDAC inhibitory activity.

In yet another aspect, the invention relates to a method for inhibiting HDAC activity in a subject, comprising administering to said subject a compound having the ability to upregulate RhoB, in an amount sufficient to inhibit HDAC activity in said subject.

In a still further aspect, the invention pertains to a method of treating conditions associated with abnormal HDAC activity in a subject in need thereof, comprising administering to said subject a therapeutically effective amount of a compound having an ability to upregulate levels of RhoB in said subject.

The invention also provides a method for monitoring the progress of treatment of a disease associated with abnormal HDAC activity comprising monitoring message and/or protein levels of trapoxin regulated genes as disclosed herein. In one embodiment, said method comprises monitoring RhoB message and/or protein levels in a subject suffering from said disease.

### DETAILED DESCRIPTION OF THE INVENTION

In practicing the present invention, many conventional techniques in molecular biology, microbiology, and recombinant DNA are used. These techniques are well known and are explained in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

The present invention describes oligomeric antisense compounds, particularly oligonucleotides, for use in modulating the function of nucleic acid molecules encoding genes that are up and down regulated by trapoxin A, ultimately modulating the amount of gene product produced. This is accomplished by providing antisense compounds which specifically hybridize with one or more nucleic acids encoding these genes. As used herein, the terms “target nucleic acid” and “nucleic acid encoding a trapoxin A regulated gene” encompass DNA encoding a trapoxin A or p21^{waf1}-regulated gene from the list provided in Tables 3, 4, and 5; RNA (including pre-mRNA and mRNA) transcribed from such DNA, and also cDNA derived from such RNA. The specific hybridization of an oligomeric compound with its target nucleic acid interferes with the normal function of the nucleic acid. This modulation of function of a target nucleic acid by compounds which specifically hybridize to it is generally referred to as “antisense”. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of a trapoxin A and p21^{waf1}-regulated genes listed in Tables 3, 4, and 5. In the context of the present invention, “modulation” means either an increase (stimulation) or a decrease (inhibition) in the expression of a gene. In the context of the present invention, inhibition is the preferred form of modulation of gene expression for genes downregulated in the presence of an HDAC inhibitor; while stimulation is preferred for genes that are upregulated in the presence of an HDAC inhibitor. In both instances, mRNA is a preferred target.

As used herein, a “trapoxin A-regulated gene” refers to a gene whose expression detectably is modulated, either up-regulated or down-regulated by trapoxin A, including, but not limited to, genes that are up or down regulated due to HDAC inhibition through trapoxin A treatment.

As used herein “abnormal HDAC activity” includes conditions which may be characterized by either too much or, in contrast, insufficient HDAC activity, compared to normal controls.

It is preferred to target specific nucleic acids for antisense. “Targeting” an antisense compound to a particular nucleic acid, in the context of this invention, is a multistep process. The process usually begins with the identification of a nucleic acid sequence whose function is to be modulated. This may be, for example, a cellular gene (or mRNA transcribed from the gene) whose expression is associated with a particular disorder or disease state or drug treatment, or a nucleic acid molecule from an infectious agent. In the present invention, the targets are nucleic acid molecules encoding trapoxin A regulated genes. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene the expression of which is induced by trapoxin A as disclosed herein, regardless of the sequence(s) of such codons.

It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a preferred target region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. mRNA splice sites, i.e., intron-exon junctions, may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, as target regions for antisense compounds targeted, for example, to DNA or pre-mRNA.

There are a variety of ways to identify antisense oligonucleotides which will be biologically active. For a mRNA of 3000 nucleotides, there are approximately 30000 different 10-20-mers possible. It is not possible to predict a priori how many and which of the 30000 possible 10-20-mer antisense oligonucleotides for a mRNA will have useful activity; experience shows that a small number will be active. The surest method to identify the best oligonucleotide would be to synthesize all 30000 sequences and test them individually. This is a method that has been used, but in practice this would be a huge effort. There are numerous reports in literature of screening only a small selection of between 10-30 different antisense sequences that provided suitable compounds. It would be helpful to be able to predict which regions of the target RNA are open to binding of oligonucleotides, however methods to determine whether a given region of a mRNA is accessible are limited. Computer programs to predict secondary interactions of RNA are available, but are relatively primitive and unable to give reliable predictions for long polynucleotides (e.g. greater than a few hundred nucleotides). The use of enzyme mapping experiments (Lima et al., Biochemistry 31: 12055-12061 (1992)) to reveal single and double-stranded regions of a RNA have been applied, and require multiple incubations with various enzymes and several polyacrylamide gels. Mapping of the RNA accessible regions with a combinatorial library of short DNA-oligonucleotides is a recently introduced technique (Lima et al., J. Biol. Chem. 272:626-638 (1997)). After hybridization of members of the library with the RNA, RNase H is introduced to give cleavage of the formed duplexes. These regions are subsequently identified on a polyacrylamide gel. This method is also limited by the resolution of the gel, and may be that single-stranded regions located by short oligonucleotides may not offer good binding sites to longer antisense sequences.

While all of the above methods can be used to select antisense, scanning array technology gives a direct readout of the binding capacity of a complete set of antisense oligonucleotides, and appears to be a big step forward in identifying accessible regions of a RNA target for high affinity duplex formation by antisense oligonucleotides (Southern et al., Nucleic Acids Res. 22: 1368-1373 (1994)). The method itself is quite straightforward and is described in detail in a number of peer-reviewed publications (see for example Southern et al., Nucleic Acids Res. 22: 1368-1373 (1994) and Nature Biotech. 15:537-541 (1997); Sohail et al., Molecular Cell Biology Research Communications 3: 67-72 (2000); WO 95/11748). A suitable device to perform the method is described in detail in WO 98/22211 (U.S. patent application Ser. No. 09/308,095). After the synthesis of the array is complete, the result is a complete set of antisense oligonucleotides in a defined set of cells covalently bound by their 3′-ends to the surface of a suitable carrier such as hydroxylated or aminated (see Matson et al., Analytical Biochemistry 207: 306-310 (1994)) polypropylene, where the sequence in each cell differs from that of its neighbours by the nucleotide at the 3′ and the 5′ ends. The polypropylene is then used as the solid phase component in a hybridization assay with the Cy-5 labeled target mRNA. Those antisense sequences present on the surface of the polypropylene which strongly bind the target are identified from their position on the array (“hot-spots”) and are subsequently tested in the desired assays.

Once one or more target sites have been identified, oligonucleotides are chosen which are sufficiently complementary to the target, i.e., hybridize sufficiently well and with sufficient specificity, to give the desired effect, using, for example, any of the techniques mentioned above.

In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

Antisense compounds are commonly used as research reagents and diagnostics. For example, antisense oligonucleotides, which are able to inhibit gene expression with exquisite specificity, are often used by those of ordinary skill to elucidate the function of particular genes. Antisense compounds are also used, for example, to distinguish between functions of various members of a biological pathway. Antisense modulation has, therefore, been harnessed for research use.

The specificity and sensitivity of antisense is also harnessed by those of skill in the art for therapeutic uses. Antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides can be useful therapeutic modalities that can be configured to be useful in treatment regimes for treatment of cells, tissues and animals, especially humans. In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, that include but are not limited to, enhanced affinity for nucleic acid target and increased stability in the presence of nucleases.

While antisense oligonucleotides are a preferred form of antisense compound, the present invention contemplates other oligomeric antisense compounds, including but not limited to oligonucleotide mimetics such as are described below. The antisense compounds in accordance with this invention preferably comprise from about 8 to about 30 bases. Particularly preferred are antisense oligonucleotides comprising from about 8 to about 30 bases (i.e. from about 8 to about 30 linked nucleosides).

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include but are not limited to, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. Techniques for the synthesis of antisense compounds containing oligonucleotides with modified backbones or non-natural internucleoside linkages as described above may be achieved using conventional methodologies, and are familiar to one of skill in the art. Representative United States patents that teach the preparation of the above phosphorus-containing linkages include, but are not limited to, U.S. Pat. Nos. 3,687,808; 4,469,863 and 5,625050; each of which is incorporated by reference herein in its entirety.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include but are not limited to those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Synthesis of such oligonucleotides may be achieved by one of skill in the art according to conventional methods, for example, as described in U.S. Pat. Nos. 5,034,506; 5,166,315 or 5,677,439 each of which is incorporated by reference herein in its entirety.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

The Most preferred embodiments of the invention are oligonucleotides having morpholino backbone structures as described in U.S. Pat. No. 5,034,506. Also preferred are oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—] as described in U.S. Pat. No. 5,489,677, and the amide backbones as described in U.S. Pat. No. 5,602,240.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O—, S- or N-alkynyl; or O-alkyl-O-alkyl, wherein the alkyl, alkenyl and alkynyl may be substituted or unsubstituted C to C₁₀ alkyl or C₂ to C₁₀ alkenyl and alkynyl. Particularly preferred are O[(CH₂)ₙO]ₘCH₃, O(CH₂)ₙ OCH₃, O(CH₂)ₙ NH₂, O(CH₂)ₙ CH₃, O(CH₂)ₙ ONH₂, and O(CH₂)ₙON[(CH₂)ₙ CH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂ CH₂ OCH₃, also known as 2′-O—(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ ON(CH₃)₂ group, also known as 2′-DMAOE. A further preferred modification of this category is the bicyclic class of modifications known collectively as LNAs (Locked Nucleic Acids) as described in Rajwanshi et al., Angew. Chem. Int. Ed. 2000, 39, 1656-1659.

Other preferred modifications include 2′-methoxy (2′-O—CH₃), 2′-aminopropoxy (2′-OCH₂ CH₂ CH₂ NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. One of skill in the art may use conventional methods to created such modified sugar structures. Representative United States patents that teach the preparation of such modified sugar structures include, but are not limited to, U.S. Pat. Nos. 4,981,957; 5,118,800 and 5,700,920 each of which is incorporated by reference herein in its entirety.

Oligonucleotides may also include base modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B. ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2.degree. C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

One of skill in the art is able to prepare modified nucleobases according to methods that are well known in the art. For example, representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302 and 5,134,066 each of which is incorporated by reference herein in its entirety.

Another modification of the oligonucleotides of the invention involves chemically linking to the oligonucleotide one or more moieties or conjugates which enhance the activity, cellular distribution or cellular uptake of the oligonucleotide. Such moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-5-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937). Representative United States patents that teach the preparation of such oligonucleotide conjugates include, but are not limited to, U.S. Pat. Nos. 4,828,979; 4,948,882 and 5,688,941 each of which is incorporated by reference herein in its entirety.

It is not necessary for all positions in a given compound to be uniformly modified, and in fact more than one of the aforementioned modifications may be incorporated in a single compound or even at a single nucleoside within an oligonucleotide. The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Short DNA oligonucleotides have short half-lives and have been readily degraded. Phosphorothioate oligonucelotides are much more resistant to degradation, so persist in the body and cells for longer periods of time. Furthermore, phosphorothioate deoxy oligo nucleotides do not stimulate RNase H, where they form duplexes with RNA. Consequently, comparable results can often be obtained with shorter oligonucleotides when chimeric oligonucleotides are used, compared to phosphorothioate deoxyoligonucleotides hybridizing to the same target region.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. One of skill in the art may prepare these hybrid structures according to conventional methods. Representative United States patents that teach the preparation of such hybrid structures include, but are not limited to, U.S. Pat. Nos. 5,013,830; 5,149,797 and 5,700,922, and each of which is incorporated by reference herein in its entirety.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The antisense compounds of the invention are synthesized in vitro and do not include antisense compositions of biological origin, or genetic vector constructs designed to direct the in vivo synthesis of antisense molecules. The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption and conventional methods for so doing exist and are familiar to one of skill in the art. For example, representative United States patents include, but are not limited to, U.S. Pat. Nos. 5,108,921; 5,354,844 and 5,595,756, each of which is incorporated by reference herein in its entirety.

The antisense compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents. The term “pharmaceutically acceptable salts” refers to physiologically and pharmaceutically acceptable salts of the compounds of the invention: i.e., salts that retain the desired biological activity of the parent compound and do not impart undesired toxicological effects thereto. Such compounds may be prepared according to conventional methods by one of skill in the art. (Berge et al., “Pharmaceutical Salts,” J. of Pharma Sci., 1977, 66, 1-19).

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl)phosphate] derivatives according to the methods disclosed in WO 93/24510 to Gosselin et al., published Dec. 9, 1993 or in WO 94/26764 to Imbach et al.

The antisense compounds of the present invention can be utilized for diagnostics, therapeutics, prophylaxis and as research reagents and kits. For therapeutics, an animal, preferably a human, suspected of having a disease or disorder which can be treated by modulating the expression of one or more trapoxin A regulated genes is treated by administering antisense compounds in accordance with this invention. The compounds of the invention can be utilized in pharmaceutical compositions by adding an effective amount of an antisense compound to a suitable pharmaceutically acceptable diluent or carrier. Use of the antisense compounds and methods of the invention may also be useful prophylactically, e.g., to prevent or delay infection, inflammation or tumor formation, for example.

The antisense compounds of the invention are useful for research and diagnostics, because these compounds hybridize to nucleic acids encoding trapoxin A regulated genes as disclosed in the Tables provided herein, enabling sandwich and other assays to easily be constructed to exploit this fact. Hybridization of the antisense oligonucleotides of the invention with a nucleic acid encoding a particular trapoxin A regulated gene can be detected by means known in the art. Such means may include conjugation of an enzyme to the oligonucleotide, radiolabelling of the oligonucleotide or any other suitable detection means. Kits using such detection means for detecting the level of a trapoxin A regulated gene product in a sample may also be prepared.

The present invention also includes pharmaceutical compositions and formulations which include the antisense compounds of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids, according to conventional methods, by one of skill in the art.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention.

The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone, or in combination with other agents, drugs or hormones.

The pharmaceutical compositions encompassed by the invention may be administered by any number of routes including, but not limited to, oral, intravenous, intramuscular, intra-articular, intra-arterial, intramedullary, intrathecal, intraventricular, transdermal, subcutaneous, intraperitoneal, intranasal, enteral, topical, sublingual, or rectal means. In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.). Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient. Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers may also be used for delivery. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

After pharmaceutical compositions have been prepared, they can be placed in an appropriate container and labeled for treatment of an indicated condition. For administration such labeling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art. For any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. Such information can then be used to determine useful doses and routes for administration in humans. A therapeutically effective dose refers to that amount of active ingredient, which ameliorates the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

In addition to the use of antisense, one of skill in the art may employ other conventional methods for modifying gene expression. For example, such methods may involve the use of molecules including ribozymes, triple helix DNA, RNA aptamers and/or double stranded RNA directed to an appropriate nucleotide sequence of a gene which is desirably downregulated as a method to treat a condition associated with abnormal HDAC activity. These inhibitory molecules may be created using conventional techniques by one of skill in the art without undue burden or experimentation. For example, inhibition of the expression of gene expression may be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Carr, Molecular and Immunologic Approaches, Futura Publishing Co., Mt. Kisco, N.Y.). These molecules may also be designed to block translation of mRNA by preventing the transcript from binding to ribosomes.

Ribozymes, enzymatic RNA molecules, may also be used to inhibit gene expression by catalyzing the specific cleavage of RNA. The mechanism of ribozyme action involves sequence-specific hybridization of the ribozyme molecule to complementary target RNA, followed by endonucleolytic cleavage. Examples which may be used include engineered “hammerhead” or “hairpin” motif ribozyme molecules that can be designed to specifically and efficiently catalyze endonucleolytic cleavage of gene sequences, for example, the genes shown herein to be down-regulated in the presence of Trapoxin.

Specific ribozyme cleavage sites within any potential RNA target are initially identified by scanning the target molecule for ribozyme cleavage sites which include the following sequences: GUA, GUU and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Ribozyme methods include exposing a cell to ribozymes or inducing expression in a cell of such small RNA ribozyme molecules (Grassi and Marini, 1996, Annals of Medicine 28: 499-510; Gibson, 1996, Cancer and Metastasis Reviews 15: 287-299). Intracellular expression of hammerhead and hairpin ribozymes targeted to mRNA corresponding to at least one of the genes discussed herein can be utilized to inhibit protein encoded by the gene.

Ribozymes can either be delivered directly to cells, in the form of RNA oligonucleotides incorporating ribozyme sequences, or introduced into the cell as an expression vector encoding the desired ribozymal RNA. Ribozymes can be routinely expressed in vivo in sufficient number to be catalytically effective in cleaving mRNA, and thereby modifying mRNA abundance in a cell (Cotten et al., 1989 EMBO J. 8:3861-3866). In particular, a ribozyme coding DNA sequence, designed according to conventional, well known rules and synthesized, for example, by standard phosphoramidite chemistry, can be ligated into a restriction enzyme site in the anticodon stem and loop of a gene encoding a tRNA, which can then be transformed into and expressed in a cell of interest by methods routine in the art. Preferably, an inducible promoter (e.g., a glucocorticoid or a tetracycline response element) is also introduced into this construct so that ribozyme expression can be selectively controlled. For saturating use, a highly and constituently active promoter can be used. tDNA genes (i.e., genes encoding tRNAs) are useful in this application because of their small size, high rate of transcription, and ubiquitous expression in different kinds of tissues.

Therefore, ribozymes can be routinely designed to cleave virtually any mRNA sequence, and a cell can be routinely transformed with DNA coding for such ribozyme sequences such that a controllable and catalytically effective amount of the ribozyme is expressed. Accordingly the abundance of virtually any RNA species in a cell can be modified or perturbed.

Ribozyme sequences can be modified in essentially the same manner as described for antisense nucleotides, e.g., the ribozyme sequence can comprise a modified base moiety.

RNA aptamers can also be introduced into or expressed in a cell to modify RNA abundance or activity. RNA aptamers are specific RNA ligands for proteins, such as for Tat and Rev RNA (Good et al., 1997, Gene Therapy 4: 45-54) that can specifically inhibit their translation.

Gene specific inhibition of gene expression may also be achieved using conventional double stranded RNA technologies. A description of such technology may be found in WO 99/32619 which is hereby incorporated by reference in its entirety.

Antisense molecules, triple helix DNA, RNA aptamers and ribozymes of the present invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding the genes of the polypeptides discussed herein. Such DNA sequences may be incorporated into a wide variety of vectors with suitable RNA polymerase promoters such as T7 or SP6. Alternatively, cDNA constructs that synthesize antisense RNA constitutively or inducibly can be introduced into cell lines, cells, or tissues.

Vectors may be introduced into cells or tissues by many available means, and may be used in vivo, in vitro or ex vivo. For ex vivo therapy, vectors may be introduced into stem cells taken from the patient and clonally propagated for autologous transplant back into that same patient. Delivery by transfection and by liposome injections may be achieved using methods that are well known in the art.

In addition to the importance of downregulating trapoxin induced genes as a means of treating or preventing conditions associated with abnormal HDAC activity in a subject, the data disclosed herein also indicate that certain genes are upregulated when HDACs are inhibited. For example, the profile of RNA expression in response to trapoxin A indicates the surprising finding that RhoB upregulation is associated with HDAC inhibition. RhoB, which was recently reported to suppress human tumor growth (Chen, Z., Sun, J., Pradines, A., Favre, G., Adnane, J., and Sebti, M. J. (2000). Both Farnesylated and Geranylgeranylated RhoB Inhibit Malignant transformation and Suppress Human Tumor Growth in Nude Mice. M. J. Biol. Chem. 275, 17974-17978), is one of the most significantly up-regulated genes by trapoxin A treatment. Northern analysis and Western analysis confirm that RhoB is up-regulated by TPX at both mRNA and protein levels. In order to investigate the mechanism of transcriptional regulation of RhoB by HDAC, H1299 cells are transfected with RhoB promoter driven reporter constructs and treated with trapoxin A. Furthermore, HDAC1 is found to suppress RhoB transcription. A survey of RNA expression in tumor and normal tissues reveal that RhoB expression is down-regulated compared with normal tissue in ovarian, ER-breast and colon cancers, suggesting a role for RhoB as a potential tumor suppressor and marker for diseases associated with abnormal HDAC activity.

Thus, it is contemplated herein that one may treat or prevent conditions associated with abnormal HDAC activity, including cancer or other diseases associated with abnormal cell proliferation, by inducing in a subject in need thereof the expression of genes shown to be up regulated in the Tables disclosed herein, particularly Rho B. This method may comprise the use of conventional methods familiar to one of skill in the art to induce gene expression. For example, nucleic acids comprising a sequence encoding a RhoB protein or functional derivative thereof, may be administered to promote Rho B function, by way of gene therapy. Gene therapy refers to therapy performed by the administration of a nucleic acid to a subject. In this embodiment of the invention, the nucleic acid produces its encoded protein that mediates a therapeutic effect by promoting Rho B function.

Any of the methods for gene therapy available in the art can be used according to the present invention. Exemplary methods are described below.

In a preferred aspect, the therapeutic comprises a RhoB nucleic acid that is part of an expression vector that expresses a RhoB protein or fragment or chimeric protein thereof in a suitable host. In particular, such a nucleic acid has a promoter operably linked to the RhoB coding region, said promoter being inducible or constitutive, and, optionally, tissue-specific. In another particular embodiment, a nucleic acid molecule is used in which the RhoB coding sequences and any other desired sequences are flanked by regions that promote homologous recombination at a desired site in the genome, thus providing for intrachromosomal expression of the RhoB nucleic acid (Koller and Smithies, 1989, Proc. Natl. Acad. Sci. USA 86:8932-8935; Zijlstra et al., 1989, Nature 342:435-438).

Delivery of the nucleic acid into a patient may be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see, e.g., U.S. Pat. No. 4,980,286 and others mentioned infra), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see e.g., U.S. Pat. Nos. 5,166,320; 5,728,399; 5,874,297; and 6,030,954, all of which are incorporated by reference herein in their entirety) (which can be used to target cell types specifically expressing the receptors), etc. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/20316; WO93/14188; and WO 93/20221). Alternatively, the nucleic acid can be introduced intracellularly and incorporated within host cell DNA for expression, by homologous recombination (see, e.g., U.S. Pat. Nos. 5,413,923; 5,416,260; and 5,574,205; and Zijlstra et al., 1989, Nature 342:435-438).

In a specific embodiment, a viral vector that contains the Rho B nucleic acid is used. For example, a retroviral vector can be used (see, e.g., U.S. Pat. Nos. 5,219,740; 5,604,090; and 5,834,182). These retroviral vectors have been modified to delete retroviral sequences that are not necessary for packaging of the viral genome and integration into host cell DNA. The RhoB nucleic acid to be used in gene therapy is cloned into the vector, which facilitates delivery of the gene into a patient.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Methods for conducting adenovirus-based gene therapy are described in, e.g., U.S. Pat. Nos. 5,824,544; 5,868,040; 5,871,722; 5,880,102; 5,882,877; 5,885,808; 5,932,210; 5,981,225; 5,994,106; 5,994,132; 5,994,134; 6,001,557; and 6,033,8843, all of which are incorporated by reference herein in their entirety.

Adeno-associated virus (AAV) has also been proposed for use in gene therapy. Methods for producing and utilizing AAV are described, e.g., in U.S. Pat. Nos. 5,173,414; 5,252,479; 5,552,311; 5,658,785; 5,763,416; 5,773,289; 5,843,742; 5,869,040; 5,942,496; and 5,948,675, all of which are incorporated by reference herein in their entirety.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. In a preferred embodiment, epithelial cells are injected, e.g., subcutaneously. In another embodiment, recombinant skin cells may be applied as a skin graft onto the patient. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, a RhoB nucleic acid is introduced into the cells such that it is expressible by the cells or their progeny, and the recombinant cells are then administered in vivo for therapeutic effect. In a specific embodiment, stem or progenitor cells are used. Any stem-and/or progenitor cells which can be isolated and maintained in vitro can potentially be used in accordance with this embodiment of the present invention. Such stem cells include but are not limited to hematopoietic stem cells (HSC), stem cells of epithelial tissues such as the skin and the lining of the gut, embryonic heart muscle cells, liver stem cells (see, e.g., WO 94/08598), and neural stem cells (Stemple and Anderson, 1992, Cell 71:973-985).

Epithelial stem cells (ESCs) or keratinocytes can be obtained from tissues such as the skin and the lining of the gut by known procedures (Rheinwald, 1980, Meth. Cell Bio. 21A:229). In stratified epithelial tissue such as the skin, renewal occurs by mitosis of stem cells within the germinal layer, the layer closest to the basal lamina. Stem cells within the lining of the gut provide for a rapid renewal rate of this tissue. ESCs or keratinocytes obtained from the skin or lining of the gut of a patient or donor can be grown in tissue culture (Pittelkow and Scott, 1986, Mayo Clinic Proc. 61:771). If the ESCs are provided by a donor, a method for suppression of host versus graft reactivity (e.g., irradiation, drug or antibody administration to promote moderate immunosuppression) can also be used.

With respect to hematopoietic stem cells (HSC), any technique which provides for the isolation, propagation, and maintenance in vitro of HSC can be used in this embodiment of the invention. Techniques by which this may be accomplished include (a) the isolation and establishment of HSC cultures from bone marrow cells isolated from the future host, or a donor, or (b) the use of previously established long-term HSC cultures, which may be allogeneic or xenogeneic. Non-autologous HSC are used preferably in conjunction with a method of suppressing transplantation immune reactions of the future host/patient. In a particular embodiment of the present invention, human bone marrow cells can be obtained from the posterior iliac crest by needle aspiration (see, e.g., Kodo et al., 1984, J. Clin. Invest. 73:1377-1384). In a preferred embodiment of the present invention, the HSCs can be made highly enriched or in substantially pure form. This enrichment can be accomplished before, during, or after long-term culturing, and can be done by any techniques known in the art. Long-term cultures of bone marrow cells can be established and maintained by using, for example, modified Dexter cell culture techniques (Dexter et al., 1977, J. Cell Physiol. 91:335) or Witlock-Witte culture techniques (Witlock and Witte, 1982, Proc. Natl. Acad. Sci. USA 79:3608-3612).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

In addition to the aspects discussed above, it is also contemplated herein that the differential gene expression data of the present invention may serve as a useful tool to screen for compounds with HDAC inhibitory activity and/or trapoxin analogs. For example, the pattern of gene expression seen in H1299 cells after exposure to the HDAC inhibitor trapoxin as disclosed herein can be used as a means to identify additional compounds with HDAC inhibitory activity and or trapoxin analogs by comparing this pattern with gene expression data from nucleic acid harvested from H1299 cells exposed to candidate compounds. All methodologies necessary for performing such a screening assay may be performed using conventional methods which are familiar to one of skill in the art.

It is further contemplated herein that the gene profile data of the present invention would be useful to identify those subjects for whom treatment with an HDAC inhibitor would be of therapeutic benefit. This could be achieved by using conventional methodologies to analyze the subjects mRNA for altered expression (up or down regulation) of any one or more of the gene disclosed in the tables provided herein. Various types of HDAC inhibitors are available and are known to clinicians and others of skill in the art.

Since it has been found that the RNA and protein levels of RhoB are up-regulated when a HDAC inhibitor, is applied to mammalian cells, increased message levels of this protein can thus be used as a marker for HDAC inhibition in a biological system. The up-regulation of RhoB RNA and protein levels can also be used as a marker for screening and identifying compounds that are HDAC inhibitors, for example by using a variety of available cell lines familiar to one of skill in the art, treating with an active compound and testing for up regulation of this protein marker. Once compounds with a high level of HDAC inhibitory activity are identified, these compounds can be used as therapeutic agents in mammals, including animals in veterinary medicine or humans, in need of treatment of diseases associated with abnormal HDAC activity.

In addition, in vivo analysis may involve assaying RhoB RNA or protein levels as a way of monitoring the systemic therapeutic efficacy of a selected active compound. For example, m-RNA levels of RhoB can be employed as a marker to monitor the progress of treatment of a subject with an active compound. An active compound is administered to a subject, e.g., a human patient, and the message level in a biological sample, e.g., white blood cells, from the subject is periodically checked for the altered level of the protein marker. When an active compound having the desired effect e.g. HDAC inhibition, is administered to a subject, RNA and protein levels of RhoB are up-regulated in the system of the subject.

Levels of expression of RhoB can be assayed from a biological sample, e.g., cell lysate, tissue lysate or white blood cell lysate, by any known method, including conventional techniques of RNA or protein quantitation such as Northern analysis or Western analysis respectively, or using commercially available glass chips such as those available from Affymetrix or other commercial supplier.

In addition, biological levels of RhoB protein may be detected using standard immunoassays and electrophoresis assays. For example, immunoassays can be used to detect or monitor biological levels of RhoB in a biological sample using RhoB specific polyclonal or monoclonal antibodies in any standard immunoassay format to measure RhoB. ELISA (enzyme linked immunosorbent assay) type assays and conventional Western blotting assays using monoclonal antibodies are exemplary assays that can be utilized to make direct determination of levels of the marker protein. Antibodies specific to RhoB are commercially available or may be made according to conventional methods by one of skill in the art.

While HDAC inhibitors may be useful agents for treating proliferative diseases including cancer, they may also have therapeutic usefulness for treating other conditions associated with abnormal HDAC activity including, but not limited to, conditions such as atherosclerosis, inflammatory bowel disease, host inflammatory or immune response, or psoriasis.

The present invention can be utilized to identify compounds that can inhibit HDAC activity. Various in vitro and in vivo experiments can be employed to screen potential compounds. For example, a cell culture may be treated with a compound and then assayed to determine mRNA and/or protein levels of RhoB. Up regulation of message level and/or increased protein levels of RhoB in the cell culture indicates that the compound is a HDAC inhibitor. Detection and quantification of RhoB mRNA or protein levels in a biological sample can be conducted by various conventional methods, as discussed above.

The present invention also provides a method for monitoring therapeutic efficacy of an active compound, which inhibits or regulates HDAC activities. For example, the gene expression data disclosed in the present invention, for example, the upregulation or RhoB, can be used as a clinical marker to monitor efficacy of a HDAC inhibitor compound on a patient. For example, when a HDAC inhibitor compound is therapeutically administered to a patient, a biological sample, e.g., blood or tissue, from the patient indicates increased RhoB mRNA and/or protein levels, especially in the target cells, when the compound has an inhibitory effect on HDAC and a corresponding therapeutic effect in the patient. Similarly, the use of such gene expression data, particularly RhoB, as a clinical marker can be used to optimize the dosage and the regimen of an active compound by monitoring expression levels in the subject's biological sample. Accordingly, the screening method of the present invention can be used to find a therapeutically effective compound and/or to find a therapeutically effective amount or regimen for the selected compound, thereby individually selecting and optimizing a therapy for a patient. Factors for consideration in this context include the particular condition being treated, the particular mammal being treated, the clinical condition of the individual patient, the site of delivery of the active compound, the particular type of the active compound, the method of administration, the scheduling of administration, and other factors known to medical practitioners. The therapeutically effective amount of an active compound to be administered will be governed by such considerations, and is the minimum amount necessary to prevent, ameliorate, or treat the disease. Such amount is preferably below the amount that is toxic to the host or renders the host significantly more susceptible to infections.

All publications, patents and patent applications mentioned in this specification are indicative of the level of skill of those skilled in the art to which this invention pertains. All publications, patents and patent applications cited herein are hereby incorporated by reference in their entirety.

The following examples serve to illustrate the invention but are not intended to limit it in any way.

### EXAMPLES

The following methods are employed to perform Examples 1-5 disclosed herein:

Cell Culture and Drug Treatments. The human non-small lung carcinoma cell line H1299 (ATCC, Manassas, Va.) is maintained in RPMI 1640 medium (Life Technologies, Rockville, Md.), supplemented with 10% FBS and 1% penicillin/streptomycin. H1299 cells at 60 to 80% confluence are treated with 30 nM TPX dissolved in dimethyl sulphoxide for a final concentration of 0.1% Me₂SO in cell culture medium or 0.1% Me₂SO for 0, 6, 12, and 18 h.

Tetracycline regulated-p21^{waf1}-10 cell line. H1299 cells are cotransfected by electroporation with 10 μg of a construct containing the complete p21^{waf1} coding sequence fused to a tetracycline operator sequence (p21^{waf1}-Tet^{O}) and 5 μg of a CMV-regulated expression vector containing a tetracycline repressor fused to VP16 and a neomycin resistance cassette (CMVTet^{R}VP16Neo) (constructs used commercially available, for example from Invitrogen). A clonal cell line, p21^{waf1}-10 is selected and maintained in 0.3 μg/ml geneticin and 1 μM tetracycline/1.4% ethanol (tetracycline).

Immunoblotting. p21^{waf1}-10 cells are plated at 1×10⁶ cells/10 cm dish and incubated in culture medium with 1 μM tetracycline or in absence of tetracycline for 72 h. Whole lysates are prepared in triple detergent lysis buffer (50 mM Tris, pH 8, 150 mM NaCl, 0.02% NaN₃, 0.1% SDS, 1% Nonidet P-40, 0.5% sodium deoxycholate, 100 μg/ml phenylmethylsulfonyl fluoride, 1 μg/ml aprotinin) and proteins are separated by SDS-polyacrylamide electrophoresis, electrophoretically transferred to nitrocellulose membranes, and incubated with anti-p21^{waf1} (BD Pharmingen, San Jose, Calif.). Immunoreactive proteins are visualized using ECL chemiluminescence system (Amersham Pharmacia Biotech., Piscataway, N.J.).

Detection of p21^{waf1} and RhoB protein induction in response to trapoxin A treatment. H1299 non-small lung carcinoma cells are treated with 30 nM TPX or 0.1% Me₂SO or untreated for 0, 6, 12, 24, and 48 for p21^{waf1} and 0, 8, 24 and 48 h for RhoB. Whole lysates are prepared in triple detergent lysis buffer (50 mM Tris, pH 8, 150 mM NaCl, 0.02% NaN₃, 0.1% SDS, 1% Nonidet P-40, 0.5% sodium deoxycholate, 100 μg/ml phenylmethylsulfonyl fluoride, 1 μg/ml aprotinin) and proteins are separated by SDS-polyacrylamide electrophoresis, electrophoretically transferred to nitrocellulose membranes, and incubated with anti-p21^{waf1} (BD Pharmingen, San Jose, Calif.) or anti-RhoB (Santa Cruz Biotechnologies, Santa Cruz Calif.). Immunoreactive proteins are visualized using ECL chemiluminescence system (Amersham Pharmacia Biotech., Piscataway, N.J.).

Cell proliferation assay. H1299 and p21^{waf1}-10 cells are plated at 1×10³ cells/well in a 96 well plate and either treated with 1 μM tetracycline or grown in the absence of tetracycline. These cells are incubated with a solution containing [3-(4,5-dimethylthiazol-2-yl)-5-(3-carboxymethoxyphenyl)-2-(4-sulphophenyl)-2H-tetrazolium, inner salt] and phenazine methosulfate (Promega, Madison Wis.) for 4 h on days 0, 1, 2, 3, and 4 of growth and then absorbances are read at 490 nM.

Cell cycle analysis. 2×10⁵ p21^{waf1}-10 cells are washed with medium to remove tetracycline and grown for 72 h in the absence of tetracycline. These cells are harvested, fixed in cold 70% ethanol, and stained with PI solution (70 μM propidium iodide, 38 mM sodium citrate, 20 μg/ml Rnase A). Cells are analyzed by flow cytometry on a FACsort instrument (Becton Dickinson, San Jose, Calif.).

β-Galactosidase (β-Gal) staining. p21^{waf1}-10 cells are plated at a density of 1×10⁴ cells per chamber slide. These cells are incubated in culture medium in the presence or absence of tetracycline for 24 h. Cells are stained to visualize senescence-associated β-Gal as described previously (Dimri, G. P. et al. (1995) Proc. Natl. Acad. Sci. U. S. A. 92, 9363-9367). Cells are fixed in 2% formaldehyde/0.2% glutaraldehyde, incubated with senescence-associated β-Gal stain solution (1 mg/ml 5-bromo-4-chloro-3-indolyl β-D-galactoside, 40 mM citric acid, sodium phosphate pH 6.0, 5 mM potassium ferrocyanide, 5 mM potassium ferricyanide, 150 mM NaCl, 2 mM MgCl₂) for 16 h, and then photographed at 40× magnification.

Incyte cDNA arrays. H1299 cells are treated with 30 nM TPX dissolved in Me₂SO or Me₂SO for 0, 6, 12 and 18 h. Final Me₂SO concentrations were 0.1% in cell culture medium. Total RNA is extracted using TRIZOL reagent (Life Technologies, Rockville, Md.) and purified using RNAeasy reagents (Qiagen, Valencia Calif.). Poly (A)⁺ RNA (mRNA) is prepared from total RNA using Oligotex columns (Qiagen, Valencia, Calif.) and the procedure is repeated twice to minimize contamination with ribosomal RNA. The mRNA concentrations are determined using RiboGreen RNA Quantitation kit (Molecular Probes, Eugene, Oreg.) and equal amounts of mRNA from each sample is used in labeling reactions that are performed by Synteni Division of Incyte Genomics, Inc. (Palo Alto, Calif.). Complementary DNAs are prepared by in vitro translation and labeled with Cy3-deoxyuridine triphosphate (Cy3) or Cy5-deoxyuridine triphosphate (Cy5) fluorescent nucleotides. cDNA samples from cells that are treated with Me₂SO are labeled with Cy3 and the TPX-treated samples are labeled with Cy5. Replicates were labeled with reversed fluorescent labels to accommodate biases attributable to labeling. The Cy3 and Cy5 samples from each time point are combined and hybridized to UniGem VI arrays (Iyer, V. R., et al., (1999) Science 283, 83-87). This array contains cDNAs that represent approximately 7000 human genes.

Affymetrix oligonucleotide arrays. Total RNA and Poly (A)⁺ RNA is isolated from TPX or Me₂SO-treated H1299 cells essentially as it was for Incyte cDNA arrays. Total RNA is isolated from p21^{waf1}-10 cells that are grown in the presence or in the absence of tetracycline for 6, 12 and 48 h. 1^{st} strand cDNA synthesis is performed using Superscript Choice System (Life Technologies) and a T7prFB-t₃₀ primer, 5′-AAACGACGGCACA CTTCGAAATTAATACGACTCACTATAGGGAGGCGG-t₃₀-3′ (SEQ ID NO 1). Biotinylated complementary DNA is made using the T7 Megascript system (Ambion, Austin, Tex.), substituting NTP labeling-mix containing biotinylated CTP and UTP. The labeled, purified cDNA is fragmented into <50 bp pieces at 95° C. in MgCl₂ buffer to-increase the efficiency of hybridization to 20-mer oligos on the arrays. These labeled fragments are hybridized to HU6800 arrays (Affymetrix, Santa Clara, Calif.) as described previously (Lockhart, D. J., et al., (1996) Nat. Biotechnol. 14, 1675-1680; Alon, U. et al., (1999) Proc. Natl. Acad. Sci. U.S.A. 96, 6745-6750). This array represents approximately 7000 human genes. Briefly, samples are hybridized for 16 h at 45° C. with constant rotation at 60 rpm. The arrays are washed and stained using an Affymetrix fluidics station. The stain included streptavidin-phycoerythrin (10 μg/ml; Molecular Probes, Eugene, Oreg.) and biotinylated goat anti-streptavidin (3 μg/ml; Vector Laboratories, Burlingame, Calif.).

Imaging and Data Analysis. Affymetrix arrays are scanned with a HP argon-ion laser confocal microscope, with a 488 nm emission and detection at 570 nm. Affymetrix fold changes are calculated using GeneChip 3.3 software (Affymetrix). Incyte Pharmaceutical Inc. uses a laser confocal microscope to determine the fluorescence intensities from Incyte cDNA arrays. The fluorescent images (Cy3 and Cy5) from the Incyte cDNA arrays are normalized to total fluorescence from each channel for all genes for labeling efficiency differences and considered the normalized data is used for calculation of the gene expression ratios. The effects of p21^{waf1} expression and HDAC inhibition are assessed by calculation of the fold change difference between hybridization intensities from TPX-treated cells and Me₂SO-treated cells. Similarly, the effects of p21^{waf1} expression in p21^{waf1}-10 cells are determined by calculating the fold change differences between the untreated and tetracycline-treated p21^{waf1}-10 cells. These fold changes reflect the differential gene expression in response to HDAC inhibition and p21^{waf1} expression. Only transcripts that had fold change differences of at least two on duplicate arrays and significant differential expression are considered further. Significance is measured using a proprietary noise model that determines the probability that a gene has the same expression signal in two experiments. Significant differential expression is indicated by p values that approached zero. A proprietary interactive tool is used to graphically represent expression profiles to analyze our expression data. Statistical analysis using hierarchical clustering is performed using statistical analysis software. Hierarchical clustering is used to determine relationships among arrays and transcripts based upon similarities in time-dependent expression patterns. Dendograms are generated to indicate the similarity relationships among arrays and transcripts based upon these patterns.

Northern Blotting. Total RNA is extracted from H1299 non-small lung carcinoma cells following treatment with 30 nM TPX or 0.1% Me₂SO or untreated for 6 h. RNA is separated by electrophoresis on denaturing agarose gels and transferred to nylon membranes. Membranes are hybridized with random hexanucleotide-primed ³²P-labeled DNA fragments that are prepared from the RhoB gene, using standard protocols.

Transfection and reporter gene assays. 5×10⁶H1299 cells are transfected in 6 well plates using Geneporter 2 reagent according to the manufacturers instructions (Gene Therapy Systems, San Diego, Calif.). Cotransfections are performed with wild type RhoB promoter (pGL3-RhoB5-wt) or RhoB promoter with a mutated CAAT element (pGL3-RhoB5-M-CAAT) and either flag vector or an expression vector containing flag-epitope tagged HDAC1. 48 h after transfection, cells are lysed in reporter lysis buffer and assayed for luciferase on a luminometer. The percent change in RhoB promoter activity is calculated relative to Beta-Galactosidase that is used as an internal transfection control.

### Example 1

### Characterization of p21^{waf1}-10 Cells to Study the Effects of Ectopic p21^{waf1} Expression

We derived a clonal cell line (p21^{waf1}-10) from H1299 cells that express p21^{waf1} in an inducible manner upon removal of tetracycline from the growth medium. This cell line will be deposited with the ATCC (Manassas, Va.), in compliance with the provisions of the Budapest Treaty, and will be assigned a deposit #______. Western blotting of p21^{waf1} in this cell line following tetracycline removal, demonstrated that p21^{waf1} is highly expressed and present only in the absence of tetracycline as early as 6 h. This pattern of p21^{waf1} expression is similar to p21^{waf1} expression in response to HDAC inhibition in H1299 cells. Both TPX-induced and ectopic p21^{waf1} expression was decreased at 48 h. This decrease may be associated with decreases in overall protein levels that may have occurred in response to decreases in DNA, RNA, and protein processing transcripts that were observed in response to both ectopic p21^{waf1} expression and HDAC inhibition (Tables 3 and 5). Alternatively, the decrease in p21^{waf1} could be a response to cell death.

Tetracycline removal from p21^{waf1}-10 cells also led to a time dependent decrease in growth rate over 4 days compared to H1299 parental cells or the same clone that was grown in the presence of tetracycline. Cell cycle analysis of p21^{waf1}-10 cells demonstrated a significant accumulation of cells in G₁ phase upon removal of tetracycline that increased from 53.8% to 84.5% and a decrease of cells in S phase from 32% to 3.7%, indicating that removal of tetracycline induced G₁ cell cycle arrest.

Furthermore, p21^{waf1}-10 cells exhibited a senescent phenotype 48 h following the removal of tetracycline that was indicated by flattened cell morphology and senescence-associated β-galactosidase staining.—Staining for β-Gal-associated senescence led to the accumulation of a perinuclear blue precipitate, a biomarker for senescence (Dimri, G. P. et al., (1995) PNAS USA 92:9363-67).

### Example 2

### Effects of p21^{waf1} Expression on Cellular Gene Expression

To determine the effects of ectopic p21^{waf1} expression on transcript profiles in H11299 cells, antisense biotin labelled RNA probes are prepared from p21^{waf1}-10 cells that are incubated in the presence or absence of tetracycline for 6 and 12 h and hybridized to the Affymetrix arrays listed in Table 1.

TABLE 1
Arrays used in ectopic p21 expression profiling

| Array type | Array code | Description |
|---|---|---|
| Affymetrix | 200hz1, 200hz2 vs 202hz1, 202hz2 | 6 h − Tet vs 6 h + Tet |
| Affymetrix | 203hz1, 203hz2 vs 205hz1, 205hz2 | 12 h − Tet vs |
| | | 12 h + Tet |

Pairwise comparisons of hybridization intensities on chips that were hybridized at 2 time points (6 and 12 h) with RNA that was isolated from untreated p21^{waf1}-10 cells that ectopically express p21^{waf1} and tetracycline-treated p21^{waf1}-10 cells in which ectopic expression is repressed indicated that the levels of 62 transcripts changed in response to overexpression of p21^{waf1}. Hierarchical clustering using euclidean distance and complete linkage of genes was performed to identify transcripts that had similar expression patterns or that may be transcriptionally co-regulated (differentially regulated above two fold in on two chips from the same time point).

These results indicated two clusters of transcripts, a small cluster of transcripts that were up-regulated in response to ectopic p21^{waf1} expression at 6 h, including apolipoprotein E and phospholipase D, genes that are involved lipid metabolism and a major cluster of genes that were downregulated at 6 h in response to ectopic p21^{waf1} expression. The expression of genes at the same time points suggests links among genes as parts of pathways that are regulated in parallel or are responses to a common upstream regulator.

The majority of the changes in response to p21^{waf1} expression were observed at 6 h (Table 3). At this time, p21^{waf1} expression led to downregulation of genes involved in the cell cycle, cellular proliferation, DNA replication, DNA repair mechanisms, transformation, and the chromatin complex. Transcripts that are involved in RNA processing, protein processing, stress, apoptosis and oncogenic transformation were also repressed, as well as transcripts for metabolic enzymes that are involved in the citric acid cycle, carbohydrate, and lipid metabolism. Interestingly, there were a limited number of transcripts that changed at 12 h (Table 4), suggesting that by 6 h most of the changes in transcript levels had already occurred.

TABLE 2
Effects of ectopic p21^{waf1} expression at 6 h

| | | Fold change | |
|---|---|---|---|
| | | 6 h | |
| GenBank ID | Gene Description | Exp1 | Exp2 |
| | Cell cycle | | |
| X77794 | Cyclin G1 | −5.5⁶ | −4.3⁶ |
| M25753 | Cyclin B1 | −2.3² | −3.8² |
| | Proliferation | | |
| U74612 | Hepatocyte Nuclear Factor-3α/forkhead homolog 11A | −4.0* | −3.8⁷ |
| X66899 | Ewings Sarcoma gene | −6.6⁶ | −8.9⁶ |
| HG2724-HT2820 | Oncogene TLS | −2.5¹ | −2.7⁶ |
| M94856 | Psoriasis-associated fatty acid binding protein | −2.1³ | −2.4¹ |
| L19686 | Macrophage migration inhibitory protein | −4.0⁶ | −3.8⁶ |
| | Mitosis | | |
| X54942 | CKShs2 | −2.6⁴ | −4.0³ |
| U33286 | Cellular apoptosis susceptibility gene (CAS) | −2.3⁶ | −2.5¹ |
| D63880 | KIAA0159, condensin homolog | −3.6⁶ | −2.3* |
| M37583 | H2A.Z | −2.2⁶ | −3.9⁶ |
| U63743 | Mitotic centromere-associated kinesin | −9.9⁶ | −2.3* |
| | DNA synthesis and replication | | |
| D00596 | Thymidylate synthetase | −6.6⁶ | −11.3⁶ |
| L33842 | Type II inosine monophosphate dehydrogenase | −2.6⁶ | −2.5⁶ |
| U21090 | DNA polymerase Δ, small subunit | −3.5⁶ | −3.1¹ |
| M87339 | Replication factor C | −3.0⁵ | −3.1* |
| X74330 | DNA polymerase primase α | −2.4* | −4.3³ |
| Z21507 | Elongation factor 1 Δ | −2.1* | −3.7³ |
| | Cell adhesion | | |
| | Histones | | |
| M37583 | H2A.Z | −2.2⁶ | −3.8⁶ |
| X60486 | H4 histone | −4.2⁶ | −5.5⁶ |
| | Non-histone chromosomal proteins | | |
| U28749 | HMGI-C | −2.3⁵ | −2.9³ |
| D63880 | KIAA0159, condensin homolog | −3.6⁶ | −2.3* |
| | Deacetylase complex | | |
| | Apoptosis | | |
| L32866 | Human effector cell protease receptor | −2.7⁷ | −2.3* |
| | Activates NFkB | | |
| U28749 | HMGI-C | −2.3⁶ | −2.9³ |
| | Stress-induced | | |
| HG3494-HT3688 | Nuclear Factor IL-6/CEBP-Beta | −3.2* | −3.6³ |
| M32886 | Sorcin CP-22 | −2.7¹ | −9.1⁶ |
| M94630 | hnRNP C-like protein | −2.3⁶ | −6.1⁶ |
| X66899 | Ewings Sarcoma gene | −6.6⁶ | −8.9⁶ |
| | DNA repair | | |
| M31899 | ERCC-3 | −2.2* | −2.4⁷ |
| U24169 | JTV-1 | −5.5* | −5.9⁷ |
| HG2846-HT2983 | Dihydrofolate reductase | −2.8* | −5.9* |
| U21090 | DNA polymerase Δ, small subunit | −3.5⁶ | −3.1¹ |
| M87339 | Replication factor C | −3.0⁵ | −3.1* |
| D30756 | KIAA0049 | −3.1* | −3.4¹ |
| | RNA splicing | | |
| M60784 | U1 snRNP-specific A protein | −2.9¹ | −3.9⁷ |
| L03532 | hnRNP M protein | −2.3¹ | −3.1² |
| HG1595-HT4788 | hnRNP1 (PTB1) | −2.2¹ | −2.3⁵ |
| X70944 | PTB-binding splicing factor | −2.3* | −4.8³ |
| | Ribosomal proteins | | |
| X79865 | Mitochondrial ribosomal protein Mrp17 | −2.9* | −3.9⁵ |
| HG2874-HT3018 | Ribosomal protein L39 homolog | −2.1* | −8.2⁷ |
| U28386 | hSRP1 | −2.6⁶ | −3.8⁶ |
| | tRNA charging | | |
| S80343 | Arginyl-tRNA-synthetase | −2.3⁴ | −4.5⁶ |
| L06845 | Cysteinyl-tRNA-synthetase | −3.1* | −3.3⁷ |
| | Protein processing | | |
| X98296 | Ubiquitin hydrolase | −2.6* | −5.5³ |
| D00760 | Proteasome subunit HC3 | −2.3³ | −3.5⁵ |
| X65867 | Adenylosuccinate lyase | −2.7¹ | −2.2* |
| X16396 | NAD-dependent methylene | | |
| | tetrahydrofolate dehydrogenase- | | |
| | methylenyltetrahydrofolate cyclohydrolase | −2.3² | −4.2⁷ |
| | Transformation | | |
| D90209 | TAXREB67 | −3.1⁶ | −10.2⁷ |
| U24576 | Breast tumor autoantigen LMO4 | −3.1* | −4.6¹ |
| HG4716-HT5158 | Guanidine 5′-monophosphate synthase | −3.3⁷ | −3.1² |
| | Carbohydrate metabolism | | |
| U24183 | Phosphofructokinase | −3.7* | −4.3⁸ |
| | Citric acid cycle | | |
| X02152 | Lactate dehydrogenase | −2.9⁸ | −6.7⁸ |
| L21936 | Succinate dehydrogenase flavoprotein | −2.9* | −3.7¹ |
| D90084 | Pyruvate dehydrogenase α subunit | −2.7* | −8.6⁸ |
| U59309 | Fumarase precursor | −2.6⁸ | −5.7⁸ |
| | Lipid metabolism | | |
| U00968 | SREBP-1 | −2.3¹ | −2.7¹ |
| M14200 | Diazapam binding inhibitor | −7.8⁸ | −11.7⁸ |
| M94856 | Psoriasis-associated fatty-acid binding | −2.1¹ | −2.4⁷ |
| D90209 | TAXREB67 | −3.1⁶ | −10.4⁷ |
| M12529 | Apolipoprotein E | 6.0⁸ | 8.9⁸ |
| | Not categorized | | |
| U25849 | Red cell-type low MW acid phospholipase | −2.5* | −10.1⁷ |
| X74008 | Protein ser/thr phosphatase-γ | −2.5⁸ | −10.4⁸ |
| U60644 | HU-K4, Phospholipase D homolog | 8.2⁸ | 8.7⁸ |
| U51586 | Siah binding protein | −4.6¹ | −2.0* |
| L20688 | Rho-GDI | −3.4¹ | −2.3¹ |

Significance levels (P values) are represented by superscripts *>.05, ¹<.05, ²<.01, ³<.005, ⁴<.001, ⁵<.0005, ⁶<.0001, ⁷<.00005, ⁸<.00001. Upregulated transcripts (+ numbers, ie. Transcripts that increased upon removal of tetracycline with concomitant p21^{waf1} upregulation), downregulated transcripts (− numbers, ie. Transcripts that decreased upon removal of tetracycline), experiment
replicate 1 (Exp1), experiment replicate 2 (Exp2), <2 fold change in expression levels (NC)

TABLE 3
Effects of ectopic p21^{waf1} expression at 12 h

| | | Fold change | |
|---|---|---|---|
| | | 12 h | |
| GenBank ID | Gene Description | Exp1 | Exp2 |
| X89985 | BCL7A | −4.0* | −3.0⁵ |
| X66363 | Cdc2 | −3.4¹ | −4.0⁸ |
| U60644 | Hu-K4, Phospholipase D homolog | −5.5⁷ | −4.1* |

Significance levels (P values) are represented by superscripts *>.05, ¹<.05, ²<.01, ³<.005, ⁴<.001, ⁵<.0005, ⁶<.0001, ⁷<.00005, ⁸<.00001. Upregulated transcripts (+ numbers, ie. Transcripts that increased upon removal of tetracycline with concomitant p21^{waf1} upregulation), downregulated transcripts (− numbers, ie. Transcripts that decreased upon removal of tetracycline), experiment
replicate 1 (Exp1), experiment replicate 2 (Exp2), <2 fold change in expression levels (NC)

A unique effect of p21^{waf1} expression was the inhibition of nucleotide excision repair (NER) transcripts. For example, ERCC3 was downregulated. This result supports previous observations that p21^{waf1} inhibited NER (Nunez, F, et al., (2000) FASEB J 14, 1073-1082) and that NER-associated transcripts were decreased in response to p21^{waf1} overexpression in HT1080 cells that express p53 (Ly, D. H. (2000) Science 287, 2486-2492).

In addition, p21^{waf1} expression mediated changes in other genes that are involved in mitotic checkpoint. For example, cyclin B1 and CKShs2, and a microtubule-associated kinesin were downregulated. Decreases in these genes could lead to incorrect spindle segregation during anaphase, resulting in mitotic crisis and cell cycle arrest. Moreover, changes in the arrangement of microtubules would be expected to alter cellular structure, that can lead to the flattened enlarged morphology that was observed in p21^{waf1}-10 cells in the absence of Tet and p21^{waf1} overexpression. This cellular phenotype and the dowregulation of mitotic checkpoint genes was associated with cellular senescence and aging. Specific responses to p21^{waf1} expression were inhibition of mitosis control and DNA repair mechanisms.

### Example 3

Effects of HDAC Inhibition on Cellular Gene Expression

Our previous studies demonstrated that a major effect of TPX in H1299 cells is growth arrest and p53-independent induction of the cyclin-dependent kinase inhibitor, p21^{waf1} (Sambucetti, L. C. et al., (1999) J. Biol. Chem. 274:34940-34947). Both Affymetrix and Incyte arrays listed in Table 2 were used to span the maximal number of transcriptional changes in response to HDAC inhibition.

TABLE 4
Arrays used in HDAC inhibition expression profiling

| Array Type | Array code | Description |
|---|---|---|
| Affymetrix | 167hz1, 167hz2 vs 170hz1, 170hz2 | 6 h DMSO vs 6 h TPX |
| | 168hz1, 168hz2 vs 171hz1, 171hz2 | 12 h DMSO vs 12 h TPX |
| | 169hz1, 169hz2 vs 172hz1, 172hz2 | 18 h DMSO vs 18 h TPX |
| Incyte | 0220AXUX, 0227AXUY vs 022LAXV1, 022EAXV0 | 6 h DMSO vs 6 h TPX |
| | 022SAXV2, 022ZAXV3 vs 022DAXV5, 0226AXV4 | 12 h DMSO vs 12 h TPX |
| | 022RAXV7, 022KAXV6 vs 0225AXV9, 022YAXV8 | 18 h DMSO vs 18 h TPX |

From both studies, changes in the levels of 66 transcripts were observed in response to HDAC inhibition. However, there were only 4 genes that changed both on the Incyte and Affymetrix arrays, 5′ nucleotidase, p21^{waf1}, HMGI-C and urokinase plasminogen activator receptor (UpaR). A subset of transcripts on Affymetrix oligonucleotide microarrays that met the criteria of >2 fold changes in intensity in response to trapoxin A-treatment relative to dimethyl sulphoxide-treated controls at the same time point, 72% of the transcripts were downregulated in a range of 2 to 50 fold. The remaining 27% of these transcripts were induced from 2 to 10 fold. Similarly, 68% of the transcripts that met the criteria of >2 fold changes in intensity in response to trapoxin A-treatment relative to the dimethyl sulphoxide control at the same time point were downregulated in a range of 2 to 9 fold and 32% were upregulated from 2 to 9 fold on Incyte arrays.

The changes in transcript levels in response to trapoxin A treatment are summarized in Table 5. Transcripts that decreased included enzymes involved in cell cycle, apoptosis, proliferation, DNA synthesis, the chromatin complex, RNA and protein processing, oncogenic transformation, tumor invasion, and metastasis. Transcripts that increased included cyclin-dependent kinase inhibitors, stress-induced genes, growth factors, small GTP-ases including RhoB, and spermidine/spermine N-acetyltransferase. Effects of HDAC inhibition through TPX treatment that were not seen by increased p21^{waf1} expression were downregulation of tumor-associated proteases and metastasis-associated transcripts. Additional microarray analysis indicates that while RhoB may be upregulated approximately 8 fold in H1299 cells treated with 30 nm TPX, expression of RhoA and RhoC is unchanged in these cells.

TABLE 5
Effects of HDAC inhibition by TPX

| | | Fold change | | Fold change | | Fold change | |
|---|---|---|---|---|---|---|---|
| | | 6 h | | 12 h | | 18 h | |
| GenBank ID | Gene Description | Exp1 | Exp2 | Exp1 | Exp2 | Exp1 | Exp2 |
| | Hormone receptors | | | | | | |
| D50920 | KIAA0130, thyroid receptor | NC | NC | NC | NC | −6.1* | −6.3⁵ |
| | interacting protein | | | | | | |
| L42176 | DRAL/FHL2, androgen receptor | −4.3* | −2.4¹ | −2.6* | −2.7* | −2.9 | −3.0 |
| | associated protein | | | | | | |
| | Chromatin remodeling | | | | | | |
| X03473 | Histone H1 (0) | 6.8⁴ | 10.8⁸ | 12.0⁸ | 11.9⁸ | 3.4* | 3.6* |
| AL047358 | Spermidine/spermine N1- | NC | NC | 3.8 | 2.3 | 2.3 | 3.4 |
| | acetyltransferase | | | | | | |
| U35113 | Metastasis-associated protein 1 | NC | NC | −2.1¹ | −6.5¹ | −3.9¹ | −7.3⁵ |
| | (MTA-1) | | | | | | |
| | DNA processing | | | | | | |
| L39874 | Deoxycytidylate deaminase | −5.3⁸ | −5.2⁸ | −4.0⁸ | −4.3⁸ | −3.4⁸ | −12.8⁸ |
| L16991 | Thymidylate kinase | NC | NC | −2.1⁸ | −3.2* | −2.9⁸ | −6.2⁸ |
| X55740 | 5′-nucleotidase | NC | NC | NC | NC | −7.3⁸ | −5.2⁸ |
| AW072424 | 5′-nucleotidase | −2.3 | −2.7 | −3.7 | −3.7 | −3.3 | −4.0 |
| D00596 | Thymidylate synthetase | NC | NC | NC | NC | −3.5 | −2.2 |
| | RNA processing | | | | | | |
| U30827 | Srp40 | −4.8¹ | −4.3* | NC | NC | NC | NC |
| AF052578 | RAN | NC | NC | NC | NC | −2.9 | −2.2 |
| AB011146 | KIAA0574, similar to an RNA | NC | NC | 2.7 | 2.7 | NC | NC |
| | binding protein | | | | | | |
| U15782 | Cleavage stimulation factor 3 | NC | NC | NC | NC | −2.2 | −2.4 |
| | Protein processing | | | | | | |
| X71874 | MECL-1 | NC | NC | −3.9⁸ | −6.6⁸ | −7.3⁸ | −3.9⁸ |
| U56833 | VBP-1 | NC | NC | NC | NC | −2.1* | −6.7⁵ |
| X78687 | G9 sialidase | 3.0⁸ | 3.4⁸ | 4.3⁸ | 4.5⁸ | 3.3⁸ | 2.6⁸ |
| J03909 | γ-IFN-inducible, IP-30 | 3.9⁵ | 3.9² | 4.7⁷ | 5.2⁸ | 17.1⁸ | 15.6⁸ |
| U77413 | O-linked-N-acetylglucosamine | NC | NC | NC | NC | −2.6 | −2.1 |
| | transferase | | | | | | |
| X16396 | NAD-dependent methylene | NC | NC | NC | NC | −2.7 | −2.6 |
| | teterahydrofolate | | | | | | |
| | dehydrogenase- | | | | | | |
| | methylenyltetrahydrofolate | | | | | | |
| | cyclohydrolase | | | | | | |
| | Cell cycle, G1 phase | | | | | | |
| U09579 | p21waf1 | 6.1* | 6.6* | 10.5⁸ | 7.0⁸ | 3.8* | 4.0* |
| U09579 | p21waf1 | 2.3 | 3.3 | 3.4 | 3.6 | 2.7 | 3.1 |
| | Differentiation | | | | | | |
| U31875 | Hep27 | 2.2* | 4.6* | 11.2⁸ | 12.3⁸ | 7.1⁸ | 8.3⁸ |
| X03473 | Histone H1 (0) | 6.8⁴ | 10.8⁸ | 12.0⁸ | 11.9⁸ | 3.4* | 3.6* |
| | Growth factor-associated | | | | | | |
| L03840 | Fibroblast growth factor | 2.4* | 2.7* | 3.2* | 4.9¹ | 3.7 | 4.4 |
| | receptor | | | | | | |
| AL047358 | Insulin-induced gene 1 | 2.2 | 2.8 | 7.9 | 6.4 | 2.0 | 2.9 |
| AL720570 | Hepatoma-derived growth | NC | NC | NC | NC | −2.2 | −2.0 |
| | factor | | | | | | |
| | Proliferation | | | | | | |
| X59798 | Cyclin D1 | NC | NC | −3.8⁸ | −8.8⁸ | −3.0⁸ | −4.4⁸ |
| U31556 | E2F-5 | −2.9⁸ | −4.0⁸ | −3.0⁸ | −4.6⁸ | −10.4⁸ | −6.5⁸ |
| X16707 | Fra-1 | −23.1⁸ | −25.9⁸ | −22.4⁸ | −23.5⁸ | −13.3⁸ | −13.8⁸ |
| M80244 | Human E16 | NC | NC | NC | NC | −4.4⁸ | −4.0⁸ |
| U68019 | hMAD3 | −6.3⁵ | −7.1⁸ | −4.5¹ | −4.8¹ | NC | NC |
| L20010 | Host cell factor | NC | NC | −3.9⁸ | −12.5⁸ | −3.7⁸ | −2.3⁴ |
| U79285 | Clone 23828 | −3.6 | −3.4 | −2.3 | −3.8 | NC | NC |
| | Development | | | | | | |
| M16938 | Homeobox C8 | −6.1⁶ | −2.1* | NC | NC | −4.2³ | −7.8⁵ |
| X16665 | Homeobox B2 (Hoxb2) | NC | NC | −1.9 | −4.1 | −2.8 | −1.9 |
| | Apoptosis | | | | | | |
| X89713 | Death-associated protein-5 | NC | NC | −2.1 | −2.7 | −2.5 | −2.8 |
| U25804 | Caspase-4 | NC | NC | NC | NC | −2.0 | −2.3 |
| U84214 | Defender against cell death 1 | NC | NC | 2.5 | 2.0 | NC | NC |
| A1581499 | Apoptosis inhibitor 2 | −2.4 | −2.0 | NC | NC | NC | NC |
| M58286 | TNF Receptor | NC | NC | −4.9¹ | −5.1¹ | NC | NC |
| X37546 | Inhibitor of apoptisis homolog B | NC | NC | −2.8 | −3.7 | NC | NC |
| X37547 | Inhibitor of apoptisis homolog C | −5.0 | −5.6 | NC | NC | −3.4 | −3.5 |
| | Stress | | | | | | |
| D85429 | Hsp 40 | 4.0⁸ | 3.9⁸ | 4.8⁸ | 5.3⁸ | 17.1⁸ | 15.6⁸ |
| M59828 | Hsp 70 | 3.5 | 4.1 | NC | NC | NC | NC |
| AF109161 | p35srj | NC | NC | 3.5 | 2.4 | 3.4 | 3.3 |
| U48795 | Cathelicidin antimicrobial | NC | NC | 8.5 | 8.5 | NC | NC |
| | peptide | | | | | | |
| | Transformation and TKRs | | | | | | |
| M76125 | Tyrosine kinase receptor (TKR) | NC | NC | NC | NC | −3.9² | −7.3² |
| | axl | | | | | | |
| X66029 | TKR axl | NC | NC | NC | NC | −2.8 | −2.6 |
| D31764 | Eph1B-like tyrosine kinase | −3.1* | −8.8⁸ | −3.1* | −4.2⁸ | NC | NC |
| | receptor | | | | | | |
| D16105 | Leukocyte receptor tyrosine | NC | NC | NC | NC | −5.2 | −7.1 |
| | kinase | | | | | | |
| N33982 | c-Yes-associated protein | NC | NC | −1.8 | −2.3 | −2.4 | −2.2 |
| U19261 | Epstein-Barr-induced LMP1 | −3.1¹ | −8.8⁸ | −3.1* | −4.2¹ | NC | NC |
| U28749 | High mobility group | NC | NC | −3.0⁸ | −3.4⁸ | NC | NC |
| | phosphoprotein I-C (HMGI-C) | | | | | | |
| U28749 | HMGI-C | NC | NC | −3.8 | −6.8 | −7.4 | −5.6 |
| AF109161 | p35srj | NC | NC | 3.5 | 2.4 | 3.4 | 3.3 |
| | Small GTP-ases | | | | | | |
| M12174 | RhoB | 6.2⁵ | 9.0⁸ | 5.2⁸ | 4.5⁸ | 6.2⁸ | 6.1⁸ |
| U32519 | GAP SH3 binding protein | 5.7⁸ | 5.8⁸ | 8.3⁸ | 4.3³ | 4.6⁸ | 4.2⁸ |
| AF052578 | RAN | NC | NC | 2.9 | 2.0 | 2.5 | 3.3 |
| AF012086 | RAN-binding protein 2-like | NC | NC | 9.1 | 8.3 | NC | NC |
| | Kinases | | | | | | |
| U09578 | MAKAP kinase (3 pk) | NC | NC | NC | NC | −2.5¹ | −6.2³ |
| L32976 | Mixed lineage kinase-3 | NC | NC | −2.4* | −2.4* | −5.5* | −5.7² |
| | Metastasis and invasion | | | | | | |
| X02419 | Urokinase plasminogen | −28.5⁸ | −15.1⁸ | −10.9* | −7.7² | −7.2⁵ | −8.2⁸ |
| | activator (Upa) | | | | | | |
| U09937 | Upa receptor (UpaR) | NC | NC | NC | NC | −2.1⁸ | −1.9⁸ |
| U09937 | UpaR | −32.5 | −6.5 | −19.0 | −5.0 | −5.2 | −17.6 |
| X04429 | Plasminogen activator inhibitor | NC | NC | NC | NC | −2.2 | −2.3 |
| | 1 | | | | | | |
| U35113 | Metastasis-associated 1, MTA-1 | NC | NC | −2.1 | −6.5 | −3.9 | −7.3 |
| D50525 | TI-227H | −6.0³ | −3.3¹ | −4.5³ | −3.0² | NC | NC |
| M55153 | Transglutaminase 2 | NC | NC | NC | NC | 2.1 | 2.6 |
| | Extracellular matrix and cellular | | | | | | |
| | adhesion | | | | | | |
| U03057 | Actin-bundling protein | NC | NC | 2.4⁷ | 2.0⁷ | NC | NC |
| M80899 | AHNAK nucleoprotein/ | NC | NC | −2.0 | −2.4 | NC | NC |
| | desmoykin | | | | | | |
| U14750 | Connective tissue growth factor | 3.1 | 3.6 | 5.6 | 3.5 | 3.6 | 4.5 |
| M86406 | Actinin | NC | NC | 2.1 | 2.4 | NC | NC |
| M34458 | Lamin B1 | −2.3 | −2.1 | −2.1 | −3.6 | −2.1 | −2.6 |
| NM006603 | Stromal antigen 2 | NC | NC | NC | NC | −2.1 | −2.4 |
| X95735 | Zyxin | −2.2 | −2.5 | −2.8 | −2.5 | −5.8 | −7.3 |
| X53586 | Integrin α-6 | NC | NC | NC | NC | −2.2 | −2.1 |

Significance levels (P values) are represented by superscripts *>.05, ¹<.05, ²<.01, ³<.005, ⁴<.001, ⁵<.0005, ⁶<.0001, ⁷<.00005, ⁸<.00001.
Results from Affymetrix chips (regular text), Results from Incyte chips (Italics), Upregulated transcripts (+ numbers, ie. Transcripts that increased in response to trapoxin A treatment), downregulated transcripts (− numbers, ie. Transcripts that decreased in response
to trapoxin A treatment), experiment replicate 1 (Exp1), experiment replicate 2 (Exp2), <2 fold change in expression level (NC)

HDAC inhibition led to increases in genes that regulate G₁ phase, such as p21^{waf1} and decreases in cyclin D1, and E2F-5. Previous observations have shown that cyclin D1 was inhibited in response to another HDAC inhibitor (Sandor, V., et al., (2000) Br. J. Cancer 83, 817-825). As previously shown, only a subset of E2F responsive genes, such as DHFR and cdc2 were downregulated in response to p21^{waf1} expression (Chang, B. D. et al., (2000) Proc. Natl. Acad. Sci. U.S.A. 97, 4291-4296).

Both HDAC inhibition and p21^{waf1} expression led to decreases in transcripts that modulate cellular proliferation and oncogenic transformation (Tables 3 and 5). Previously, HDAC inhibitors were shown to modulate genes that are associated with oncogenic transformation (Ly, D. H. et al., (2000) Science 287, 2486-2492) and reverted morphological changes seen following ras-induced cell transformation (Kim, M.-S. et al., (2000) Cancer Lett. 157, 23-30). HDAC inhibition decreased the expression of axl tyrosine kinase. Axl is a member of a family of transmembrane receptor oncogenes whose downregulation has been associated with growth suppression. HDAC inhibition also decreased other tyrosine kinase receptors (TKR) and one TKR-associated protein (Table 5). Therefore, HDAC inhibitors may downregulate proliferation by inhibiting growth-stimulating kinase cascades at the level of TKR transcription. Examples of p21^{waf1}-inhibited transcripts that are associated with proliferation (Table 3) and tumorigenesis included two closely related oncogenes, Ewings sarcoma gene (EWS) and TLS. Both EWS and TLS genes bind heterogeneous RNA-binding proteins (hnRNP), many of which were also downregulated. Furthermore, p21^{waf1} inhibited the expression of psoriasis-associated fatty acid binding protein and macrophage migration inhibitory factor transcripts (Kwon, H. J. et al., (1998) Proc. Natl. Acad. Sci., U.S.A. 95, 3356-3361; Inan, M. S. et al., (2000) Gastroenterology 118, 724-34), genes that are associated with psoriasis, a disease that is characterized by hyper-proliferation and inflammation. Similarly, HDAC inhibition has been found to decrease the expression of certain cytokines and NFkB that are associated with inflammatory bowel disease (Gibson, P. R. (2000) Gut 46, 447-448; Segain, J. P. et al., (2000) Gut 47, 397-403; Inan, M. S. et al. (2000) Gastroenterology 118, 724-734). These results provide evidence that p21^{waf1} and HDAC inhibition might play important regulatory roles as inhibitors of proliferative diseases.

DNA replication and chromatin structure: HDAC inhibition by TPX and p21^{waf1} expression also led to downregulation of transcripts that are involved in DNA replication (Table 3 and 5). HDAC inhibition downregulated predominantly nucleotide scavenging enzyme transcripts. These enzymes have been used as surrogate markers of tumor growth, further elucidating a role for HDAC in tumor cell proliferation. p21^{waf1} expression also downregulated transcripts that control DNA unwinding, polymerization, and elongation. Similarly, HDAC has been shown to have effects on DNA replication factor protein complexes. For example, HDAC1, HDAC2 and topoisomerase II were found to modify each others activity. Furthermore, transcripts that regulate nucleosome assembly, including histones and non-histone chromosomal proteins that are involved in chromatin compaction were decreased in response to HDAC inhibition. These effects would be expected to decrease DNA replication and impose a block on transcription. HDAC inhibition by TPX led to changes in genes that are associated with HATs, hormone receptors, and nucleosome-remodeling complexes that have been shown to be associated with HDACs. For example, HDAC inhibition through TPX treatment led to increases in spermidine/spermine-N 1-acetyltransferase. Nucleosome remodeling complex-associated factors that were downregulated included metastasis-associated protein 1 (MTA-1) that was found to be associated with HDAC1 and histone variant H2A.Z was downregulated (Toh, Y. et al. (2000) J. Exp. Clin. Cancer. Res. 19, 105-11; Xue, Y. et al., (1998) Mol. Cell. 2, 851-861; Sah N. K. et al., (1999) Proc. Natl. Acad. Sci. U.S.A. 96, 4838-4843).

Protein processing: HDAC inhibition through TPX treatment led to decreases in transcripts that are involved in protein degradative processes and increases in protein-protective genes (Table 5). Similarly, p21^{waf1} expression inhibited both transcripts that are involved in protein synthesis and turnover (Table 3). For example, HDAC inhibition through TPX treatment repressed proteasomal genes, such as proteasome subunit-like MECL-1, VHL-binding protein, and genes that are associated with the major histocompatibility complex that are involved in antigen processing, such as G9 sialidase and γ-IFN-inducible IP-30 (GILT). Furthermore, HDAC inhibition through TPX treatment increased stress-induced protective proteins such as, Hsp 40, Hsp 70, and p35 that have been associated with resistance to apoptosis. HDAC inhibition through TPX treatment also led to downregulation of other pro-apoptotic and anti-apoptotic transcripts (Table 5), which may explain our previous observation that H1299 cells do not undergo apoptosis in response to TPX-induced HDAC inhibition (Sambucetti et al., (1999) J. Biol. Chem. 274:34940-47).

There was limited overlap in the genes altered in response to both HDAC inhibition through TPX treatment measured on Affymetrix chips and p21^{waf1} expression that included changes in the levels of p21^{waf1}, HMGI-C, NAD-dependent methylene tetrahydrofolate deydrogenase-methyltetrahydrofolate cyclohydrolase. These results suggested that HDAC inhibition had parallel p21^{waf1}-independent effects on gene expression that are represented by the genes changed in response to HDAC inhibition that did not overlap. Similarly, there were effects on gene expression that were unique to ectopic p21^{waf1} expression. In addition, non-specific effects may have been observed in response to the alkylating activity of TPX and solvents Me₂SO and ethanol that were used as vehicles for TPX and tetracycline, respectively.

Several cellular pathways were modulated uniquely by either HDAC inhibition by TPX treatment or p21^{waf1} expression. A unique effect of HDAC inhibition through TPX treatment was changes in the transcript levels of genes that are associated with cell adhesion and tumor invasiveness. HDAC inhibition through TPX treatment led to decreases in extracellular matrix (ECM) proteases and metastasis-associated transcripts including, urokinase plasminogen activator (Upa), Upa receptor, plasminogen activator inhibitor-1, MTA-1, TI-227H, and transglutaminase 2. Although Upa and PAI-1 were both reported as upregulated in response to p21^{waf1} overexpression in HT1080 cells that express p53 (Sandor, V. et al., (2000) Br. J., Cancer 83:817-825), there were no changes found in these transcripts in p53-deficient H1299 cells. HDAC inhibition through TPX treatment may inhibit tumor invasion by regulating the cellular levels of ECM degrading enzymes and metastasis-associated genes. Therefore, in addition to inhibiting tumorigenesis by inducing cell cycle arrest and apoptosis, HDAC inhibition through TPX treatment may also control later events that determine tumor invasiveness.

### Example 4

### Northern Analysis Confirmed Changes in Transcript Levels in Response to HDAC Inhibition by TPX Treatment

A subset of genes on Incyte cDNA arrays that were differentially expressed in response to HDAC inhibition by TPX treatment were analyzed using Northern blot analyses to confirm that the level of these transcripts changed in response to HDAC inhibition. Total RNA that was isolated from TPX-treated H1299 cells for 0, 6 and 18 h was hybridized with probes that were prepared from sequences of 8 differentially expressed transcripts, including G9 sialidase, IPP isomerase, p21^{waf1}, Interferon γ-inducible IP-30 (GILT), Hsp 70, urokinase plasminogen activator receptor, and KIAA0797. Although there were differences in the absolute changes in transcript levels that were determined by Northern analysis versus the arrays, the directional trend in the differential expression in response to TPX was confirmed for all of the genes tested.

### Example 5

### Methods for Detection of RhoB in Response to HDAC Inhibitors

Detection of RhoB by Immunoblot:

H1299 non-small lung carcinoma cells are treated with 30 nM trapoxin A or 0.1% dimethysulphoxide or untreated for 0, 8, 24 and 48 h for RhoB. Whole lysates are prepared in triple detergent lysis buffer (50 mM Tris, pH 8, 150 mM NaCl, 0.02% NaN₃, 0.1% SDS, 1% Nonidet P-40, 0.5% sodium deoxycholate, 100 μg/ml phenylmethylsulfonyl fluoride, 1 μg/ml aprotinin) and proteins are separated by SDS-polyacrylamide electrophoresis, electrophoretically transferred to nitrocellulose membranes, and incubated with anti-RhoB (Santa Cruz Biotechnologies, Santa Cruz Calif.). Immunoreactive proteins are visualized using ECL chemiluminescence system (Amersham Pharmacia Biotech., Piscataway, N.J.).

Detection of RhoB by Northern Analysis:

Total RNA is extracted from H1299 non-small lung carcinoma cells following treatment with 30 nM TPX or 0.1% Me₂SO or untreated for 6 h. RNA is separated by electrophoresis on denaturing agarose gels and transferred to nylon membranes. Membranes are hybridized with random hexanucleotide-primed ³²P-labeled DNA fragments that are prepared specifically against the RhoB gene, using standard protocols.

DNA microarray analysis indicates that the small GTPase RhoB, is a gene that is among a small set of genes that are strongly up-regulated in response to trapoxin A treatment. Northern and Western analysis confirm that RhoB is up-regulated by trapoxin A at both mRNA and protein levels. To further investigate the mechanism of transcriptional regulation of RhoB by HDAC1, H1299 cells are cotransfected with a flag epitope tagged HDAC1 expression vector and either a wild type RhoB promoter driven luciferase reporter construct (pGL3-RhoB5-wt) or a RhoB luciferase reporter construct containing a mutated CAAT element (pGL3-RhoB5-M-CAAT) prepared according to conventional methods. Data indicate that HDAC1 specifically suppress the activity of the wild type but not the CAAT element mutated RhoB promoter. Our results demonstrate that the small GTPase RhoB is a target of trapoxin A, and that HDAC represses RhoB transcription through an inverted CAAT element in its promoter. These findings shed light on the mechanisms of HDAC-mediated transcriptional repression and suggest that RhoB may be a useful biomarker in diseases associated with abnormal HDAC activity.

### Example 6

### Synthesis of Oligonucleotides

Modified synthetic oligonucleotides described in this invention may be prepared using standard phosphoramidite chemistry on ABI394 or Expedite/Moss Synthesizers (Applied Biosystems, Foster City, Calif.). Phosphoramidites are dissolved in acetonitrile at 0.05 M concentration, coupling is made by activation of phosphoramidies by a 0.2 M solution of benzimidazolium triflate in acetonitrile. Coupling times are usually between 3-6 minutes. A first capping is made using standard capping reagents. Sulfurization is made by using a 0.05 M solution of N-ethyl, N-phenyl-5-amino-1,2,4-dithiazol-3-thione for two minutes (Described in EP-A-0992506). Oxidation is made by a 0.5 M solution of t-butyl hydroperoxide in toluene for two minutes. A second capping is performed after oxidation or sulfurization. Oligonucleotide growing chains are detritylated for the next coupling by 2% trichloroacetic acid. After completion of the sequences the support-bound compounds are cleaved and deprotected as “Trityl-on” by 32% aqueous ammonia at 80° C. for two hours.

The obtained crude solutions are directly purified by RP-HPLC. The purified detritylated compounds are analyzed by Electrospray Mass spectrometry and Capillary Gel Electrophoresis and quantified by UV according to their extinction coefficient at 260 nM.

Using conventional methods, backbone and 2′-ribose modifications may be introduced into the synthesized antisense-oligonucleotides,

### Example 7

### Preparation of a Scanning Array for the Selection of Antisense Oligonucleotides Against Trapoxin Down-Regulated Genes by the Scanning Array Method

Preparation of a scanning array for the selection of antisense oligonucleotides against Trapoxin down-regulated genes may be performed as indicated below:

Preparation of the Array:

A sheet of natural rubber (400 mm×300 mm×0.75 mm) (Richterich & Zeller, Basel, Switzerland) is fitted to the array maker (Hall et al. WO 98/22211, U.S. patent application Ser. No. 09/308,095) between the hydroxylated polypropylene (PP) sheet (400 mm×300 mm×0.04 mm) prepared as is described in Friedrich et al. (Adhaes. Kleben Dichten 41: 28, 30-33 (1997)), and steel support plate of the apparatus. Four identical steel square reaction chambers (21.6 mm×10 mm×1 mm deep) are employed, each reaction chamber is fitted with 2×42 N springs to ensure a good seal to the polypropylene sheet. Each reaction chamber is connected to the inlet/outlet tubing of an ABI 394 DNA synthesiser (Perkin Elmer, Foster City, USA). The displacement of the chambers after each coupling is programmed to 1.2 mm. This produces a scanning array of DNA antisense oligonucleotides of 18-mers in length. The DNA synthesiser is programmed to synthesize simultaneously 4 sequences (1 sequence for each reaction chamber) in columns 1, 2, 3 and 4. Commercially available DNA-phosphoroamidites are used (5′-O-Dimethoxytrityl-N-2-isopropylphenoxyacetyl-2′-deoxyguanosine-3′-O-(beta-cyanoethyl-N,N,-diisopropyl) phosphoramidite; 5′-O-Dimethoxytrityl-thymidine-3′-O-(beta-cyanoethyl-N,N,-diisopropyl) phosphoramidite; 5′-O-Dimethoxytrityl-N-6-benzoyl-2′-deoxyadenosine-3′-O-(beta-cyanoethyl-N,N,-diisopropyl) phosphoramidite; 5′-O-Dimethoxytrityl-N-4-benzoyl-2′-deoxycytodine-3′-O-(beta-cyanoethyl-N,N,-diisopropyl) phosphoramidite, all from Amersham Pharmacia Biotech, Uppsala, Sweden) and the first base (3′-end) in each sequence is coupled in the uppermost position of the PP sheet and subsequent bases are added as the chambers move vertically down the sheet. The capping step of a standard oligonucleotide synthesis protocol is omitted. A standard synthesis protocol for DNA synthesis is used: phosphoramidites are dissolved in acetonitrile at 0.04 M concentration, coupling is made by activation of phosphoramidites by a 0.5 M solution of tetrazole in acetonitrile. Coupling times are 40 seconds. Oxidation is made using an aqueous iodine solution (15 g of iodine dissolved in a 1 litre 9:1 solution of acetonitrile:lutidine containing 20 ml water). Coupled nucleosides are detritylated for the next coupling using 2% dichloroacetic acid dissolved in 1,1,1-trichloroethane). After detritylation the reaction chambers are advanced to the next position.

Each time that the chambers moved one step, a needle punched a small hole in the polypropylene foil. The size of the hole is approximately 100 micron. The center of the hole is at the same horizontal level as the center point of the reaction chamber. Each hole served to reference the exact position of the reaction chamber for every synthetic coupling step, and therefore also for every oligonucleotide. In this way, each hole in the PP is associated with a particular 18-mer antisense oligonucleotide sequence. The hole can be seen with the naked eye, and is also detectable during the image analysis after the hybridization and scanning procedure. Small amounts of Cy5-labeled sample bound strongly at the punched hole.

Oligonucleotide Deprotection of the Array:

The polypropylene sheet is removed from the array maker and rinsed in isopropanol. It is then immersed in 32% aqueous ammonia in the circular reaction drum at room temperature. The drum is rotated slowly for 16 h to ensure good reagent mixing. The foil is removed from the drum, rinsed with ethanol and dried briefly in air before use.

Preparation of the Labelled Target mRNA:

In vitro transcription (25 μg linearized with Not I) is done starting from cDNA clone using the RIBO MAX T7 kit from Promega (Madison Wis.). For labelling the mRNA of a trapoxin regulated gene, 500 μg RNA in 55 μl water may be oxidized into dialdehyde by adding 8 μl freshly prepared 100 mM aqueous sodium periodate followed by an 1 h incubation at room temperature in the dark. The excess of oxidant is removed by adding 5 μl of a 200 mM solution of sodium sulfite and incubation for 20 min at room temperature. After adding 300 μl of 50 mM sodium acetate buffer pH 4, 60 μl of 20 mM aqueous ethylenediamine hydrochloride pH 7.2 is added to the oxidized RNA. The reaction mixture is incubated for 1 hr at 37° C., and the aldimine bond between the RNA and the spacer is stabilized by reduction with 30 μl freshly prepared 200 mM sodium cyanoborhydride in acetonitrile. Incubation takes place for 30 min. at room temperature and precipitation is done with 2 volumes of 2% lithium perchlorate in acetone for 1 hour at −20° C. The sample is spun at 14000 rpm for 45 min (3-4° C.) and after removing of the supernant the RNA pellet is washed twice with acetone and air dried. Conjugation is carried out by resuspending the amino modified RNA in 100 μl DEPC-treated water and adding 400 μl of 1 mM Cy5 N-hydroxysuccinimidyl active ester in 1 M sodium phosphate buffer (pH 7.8). After incubation for 30 min at room temperature the mixture is applied to a Quiagen column (“RNEASY Midi”) following the RNA purification protocol described by the manufacturer. 250 μl fractions (2×) are collected and the quality and amount of labelled RNA checked on a 0.8% agarose gel (TAE buffer, 80 V, 1 hr). Visualization of the bands takes place by ethidium bromide staining and by using the STORM™ red diode laser (Molecular Dynamics, Sunnyvale, Calif.).

Scanning Array Hybridization with Labeled mRNA Transcript:

After synthesis of the scanning array polypropylene foil is complete, the sheet is prewashed with 50 ml hybridization buffer (10 mM phosphate, pH 7.2, 180 mM KCl, 20 mM NaCl, 0.1 mM EDTA, 0.1% Tween-20, 1 mM MgCl2, pH 7.0, for 60 min at 37° C. in a Plexiglas barrel. The Cy5-labelled RNA (99 μg) is hybridized in 50 ml hybridization buffer at 37° C. for 1 hr, rinsed with a cold solution containing 1 M NaCl, 0,1% Tween and 1 mM MgCl2. Image analysis is carried out with an 860 STORM™ (red diode laser scanning) image analyzer and the AIS software (version 3.0: Imaging Research Inc., Ontario, Canada). An image grid is constructed using the software of 2 parallel columns of 201 cells, 1.7 mm in diameter. During image analysis one column of the grid is fitted exactly over the needle holes of the image: the other column of cells thus fits exactly over the area on the PP sheet of each surface-bound 18-mer antisense oligonucleotide in the array columns, such that each needle hole is associated with an identifiable separate 18-mer antisense oligonucleotide. The relative binding intensity of each 18-mer antisense oligonucleotide sequence in the 4 columns of the scanning arrays is measured, the exact nt sequence of each 18-mer antisense oligonucleotide is identified via cross reference to its associated needle hole.

Those sequences which show enhanced binding to the target mRNA may then be selected and synthesized as modified synthetic oligonucleotides for biological assays.

### Example 8

### Real Time Quantitative PCR Analysis of Antisense Oligonucleotides Against Selected Gene Targets

Real time quantitative PCR analysis of antisense oligonucleotides against selected gene targets disclosed herein may be achieved according to the techniques described below:

For example, H1299 cells may be grown in RPMI 1640 medium (Life Technologies, Rockville, Md. #21875-034) supplemented with 10% bovine calf serum (BCS) (Life Technologies, #16170-086) in a 5% humidified CO₂ atmosphere at 37° C.

For real time Q PCR assays one day prior to the transfection 2×10⁵ H1299 cells/well may be plated in 24 well assay plates. Oligonucleotides are stored at 100 μM concentration in TE (10 mM Tris pH 8.0, 1 mM EDTA). All oligonucleotides are diluted in OPTIMEM-I (Life Technologies, Rockville, Md.) 125-fold (0.8 μM). Separately, LIPOFECTIN (1 mg/ml, Life Technologies, Rockville, Md.) is diluted 83.3-fold in OPTIMEM-I (12 μl/ml) and left at room temperature for 30 min. A 1:1 mixture with the final concentration (400 nM for the oligonucleotides; 1.5 μl/ml LIPOFECTIN/100 nM oligonucleotide) is prepared and left for 15 min. before adding to the cells after medium has been aspirated. Cells are transfected for 4 h. After transfection the culture medium is aspirated, 0.4 ml RPMI 1640 medium containing 10% bovine calf serum is added, and the cells are incubated in 5% humidified CO₂ atmosphere at 37° C. for 20 h. Total RNA from treated as well as untreated H1299 cells may be prepared using the RNAEASY MINI kit (Qiagen) for the isolation of total RNA from animal cells following the manufacturer's instructions. The RNA is recovered in 100 μl Rnase-free H₂O and the RNA concentration is determined by measuring the fluorescence after adding the RIBO GREEN RNA quantification dye (Molecular Probes, Eugene, Oreg.) in a Victor-2 fluorometer (Wallac, Turku, Finland). Primers and TAQ MAN probes for real time PCR (containing 5′ FAM fluorescent reporter and 3′ TAMRA quencher dyes) may be purchased from BIG (Freiburg, Germany). Sequences are selected using the Primer Express Software (Perkin Elmer) at default settings.

RT real time PCR may be performed using the TAQ MAN PCR Core reagent kit (Perkin Elmer) in a ABI GenAmp 5700 thermocycler (PE Biosythems Foster City, CA) following the manufacturer's instructions. In short, 50 ng total RNA in 12 μl Rnase-free H₂O may be prepared in MicroAmp optical 96 well reaction plates (Perkin Elmer) together with 13 μl TaqMan PCR reagent mix as provided above.

### Example 9

### Cell Culture and Antisense Transfection Protocol for Genes Regulated by Trapoxin A Treatment According to Q-PCR

An antisense growth assay for a trapoxin regulated gene of the present invention may be performed as described below:

Human A549 non-small lung carcinoma cells (ATCC CCL-185) and T24 bladder carcinoma cells (ATCC HTB-4) may be cultured in DMEM medium (4.5 g/l glucose; Life Technologies), human H1299 non-small lung carcinoma cells (ATCC CRL-5803) in RPMI 1640 medium (Life Technologies), both containing 10% fetal calf serum (FCS, Life Technologies) plus 50 μg/ml Gentamycin (Life Technologies) in a humidified incubator with a 5% CO₂ atmosphere at 37° C.

Transfection of oligonucleotides may be performed using LIPOFECTIN (Life Technologies) as the carrier. Prior to transfection, cells may be plated at a density of 1.5×10³ cells/well in 200 μl medium in 96 well plates (Costar #3610 white plate with clear bottom) and grown up for 24 h.

For transfection of oligonucleotides a transfection mix may be prepared in OPTIMEM (Life Technologies) containing 1.5 μl/ml LIPOFECTIN per 100 nM oligonucleotide. Prior to the transfection, LIPOFECTIN (1 mg/ml, Life Technologies) is diluted to 486 μg/ml in OPTIMEM (Hepes buffered cell culture medium containing insulin and transferrin (Life Technologies) in a total volume of 50 μl [LIPOFECTIN transfection mix=24.3 μl LIPOFECTIN+25.7 μl OPTIMEM in 1.5 ml micro test tubes (Eppendorf)] and incubated at room temperature for 30 min. Oligonucleotides are prediluted to 32 μM in OPTIMEM in a total volume of 50 μl [oligonucleotide transfection mix=16 μl oligonucleotide (100 μM)+34 μl OPTIMEM] in row A of a 96 well plate and incubated for 10 min at room temperature. Subsequently, 50 μl of the LIPOFECTIN transfection mix is added, mixed with the oligonucleotide transfection mix of row A and incubated at room temperature for 10 min. to allow formation of the oligonucleotide/LIPOFECTIN complex. Finally, 200 μl OPTIMEM per well is added.

A deep-well multiplate (capacity 2.2 ml/well) may be prepared containing 1500 μl OPTIMEM in row A and 600 μl OPTIMEM per well in rows B to H. The 300 μl oligonucleotide/LIPOFECTIN complex is added to the 1500 μl OPTIMEM in row A resulting in a concentration of oligonucleotides of 900 nM and of LIPOFECTIN of 13.5 μl/ml. By repeatedly transferring 1200 μl to 600 μl of the next row a ⅔ dilution series is achieved with oligonucleotide concentrations of 900 nM (row A), 600 nM (row B), 400 nM (row C), 267 nM (row D), 178 nM (row E), 118 nM (row F) and 79 nM nM (row G). No oligonucleotide is given to the last row (0 nM oligonucleotide in row H). Finally, each well contains 600 μl of transfection mix which can be used to transfect 6 wells of a 96-well multiplate.

For transfection the standard medium may be removed from the cells grown in the 96-well multiplate. Of the transfection mix 90 μl are added to the cells (OPTIMEM only as control) and incubated for 4 h at 37° C. in a 5% CO₂ incubator. After incubation the transfection mix is removed and replaced with standard growth medium and the cells are incubated at 37° C. in a 5% CO₂ incubator.

Four days after the transfection the cells may be subjected to two cell proliferation assays: the WST-1 assay (Roche Molecular Biochemicals) may be used to measure the metabolic activity number of the cells, whereas the CyQuant Assay (Molecular Probes Eugene, Oreg.) may be used to measure the nucleic acid (primarily DNA) content of the cells regardless whether at this point these cells are alive or dead.

WST-1 Staining for Cell Viability and Proliferation:

The analysis of H1299 and A549 cell viability and proliferation may be performed in 96 well plates. Four days after transfection the medium is removed from the wells and replaced by 100 μl medium containing 10% WST-1 (Roche Molecular Biochemicals Indianapolis, Ind.). After 1 h (for A549 cells) or 2 h (for H1299 and T24 cells) incubation at 37° C. in a 5% CO₂ incubator the absorbance at 450 nm is measured using a microplate reader (Spectramax 250, Molecular Devices, Sunnyvale, Calif.).

CyQUANT Cell Proliferation Assay:

For the analysis of H1299 and A549 cell proliferation using the CyQUANT assay kit (Molecular Probes), medium/WST-1 may be removed from the wells and cells rinsed with 200 μl PBS. After addition of 50 μl special lysis buffer (50 mM Tris, pH 7.8; 2 mM EDTA; 1% Triton X-100) plates are frozen at −80° C. until samples are to be assayed. For monitoring, the plates are thawed at room temperature and 150 μl CyQUANT reagent (CyQUANT GR dye diluted 1:300 in 1.3× lysis buffer (included in the kit) added to each well. Sample fluorescence may be measured using a Titertek Fluoroskan II (BioConcept, Boston, Mass.) fluorescence microplate reader at 485 nm excitation and 535 nm emission maxima.

The following methods are employed to perform Examples 10-13 below:

Cell Culture, Transfection and Reporter Assay.

The human non-small lung carcinoma cell line, H1299 and the human colon carcinoma cell line, HCT116, are cultured in RPMI 1640 (Life Technologies, Gaithersburg, Md.), while the HeLa cell line is maintained in DMEM medium (Life Technologies). All media is supplemented with 10% FBS and 1% penicillin/streptomycin (Life Technologies). The cells are incubated at 37° C. with 5% CO₂. TPX is added directly to the cell medium at a final concentration of 30 nM. Transfections are performed using GenePORTER 2 (Gene Therapy Systems, San Diego, Calif.) or FuGENE 6 (Roche, Indianapolis, Ind.) transfection reagents according to the manufacturer's instructions. To normalize for transfection efficiency, cells are transfected with a luciferase construct and divided into two groups, one group is treated with 30 nm TPX, and the other is treated with same amount of DMSO (TPX vehicle) for 24 h, respectively. Luciferase activity from TPX-treated cells is normalized to luciferase activity from DMSO-treated cells. For the HDAC co-transfection experiment, cells are co-transfected with luciferase construct and pCMV-β-gal. In this case, luciferase activity is normalized to the β-gal activity.

Microarray Analysis.

Microarray analysis is performed essentially as described in Welsh, J. B. Cancer Res. 61 5974-8 (2001). Poly (A)+ RNA is prepared from H1299 cells treated with TPX or DMSO for 0, 6, and 24 h. RNA is also prepared from 136 transformed cell lines. Labelled cDNA is prepared and hybridized to HU6800 or U95a oligonucleotide arrays (Affymetrix, Santa Clara, Calif.). The arrays are scanned on an Affymetrix confocal scanner and analyzed with GENECHIP 3.1 software (Affymetrix). T-test of the results is done.

Northern and Western Analysis.

Total RNA is isolated from H1299 cells treated for 6 h with 30 nM TPX or DMSO using Trizol reagent (Life Technologies). The RNA samples are analyzed by Northern blots using conventional methods. A full-length cDNA of p21^{waf1} is used as a probe. Specific probes for RhoA, RhoB and RhoC are amplified by RT-PCR and used for hybridizations. To ensure equal loading, the β-actin levels are used as a loading control. The primers used for probe amplification are the following:

| | |
|---|---|
| RhoA-forward | |
| 5′-GTCCTTTTGACACTGCTCTAAC-3′ | (SEQ ID NO 2) |
| RhoA-reverse | |
| 5′-CTTGAGATGACACTGCTCTAAC-3′ | (SEQ ID NO 3) |
| RhoB-forward | |
| 5′-TCAGATGTTCGCCCTTCACCAG-3′ | (SEQ ID NO 4) |
| RhoB-reverse | |
| 5′-GTTACAGCGTACAAGTGTGGTCAG-3′ | (SEQ ID NO 5) |
| RhoC-forward | |
| 5′-TCACAGGGGTACAGAAATTATCC-3′ | (SEQ ID NO 6) |
| RhoC-reverse | |
| 5′-GACCAAATGCAGTGAGAGACAAG-3′ | (SEQ ID NO 7) |

Total protein is extracted from H1299 cells at 0, 6, 24 and 48 h following TPX treatment and subjected to Western analysis using an antibody against RhoB (Santa Cruz, Calif.) according to conventional methods.

Plasmid Preparation.

To identify the 5′-upstream region of the human RhoB gene, GenBank and Incyte LifeSeq databases were searched to identify RhoB cDNAs with the longest 5′-end extensions. The cDNAs were subsequently mapped to the human genome. 1 kb genomic regions upstream of the cDNA start sites were selected as the putative promoter. A 0.97 kb human RhoB promoter fragment corresponding to a region from −253 to −1224, relative to the translation initiation site (+1 ATG), was amplified. The series of deletion fragments of the RhoB promoter is made by amplifying specific 5′-upstream sequences that are inserted into the pGL3 basic vector (Promega, Madison, Wis.) which contains a luciferase reporter gene. These constructs are designated as pGL3RhoB1 (−1124/−253), pGL3RhoB2 (−821/−253), pGL3RhoB3 (−557/−253), pGL3RhoB4 (−497/—392) and pGLRhoB5-WT (−497/−392). The RhoB5 promoter fragments containing point mutations or insertions are used to generate pGL3RhoB5-M-CAAT, pGL3RhoB5-M-TATA, pGL3RhoB5-M-CATA, and pGL3RhoB5-M-Ins. The full-length cDNAs of RhoA and RhoB are obtained by reverse transcription-polymerase chain reaction (RT-PCR) from mixed human tissue mRNA (Clontech, Palo Alto, Calif.). The fragment is inserted into the BglII/HindIII sites of the pcDNA3.1 expression vector (Invitrogen, Carlsbad, Calif.) to produce pcDNA-RhoA and pcDNA-RhoB. The primers that are used for amplification are:

| | |
|---|---|
| RhoA-cDNAst: | |
| 5′-ATC AAG ATC TAT GGC TGC CAT CCG | (SEQ ID NO 8) |
| GAA GAA ACT GGT G-3′, | |
| RhoA-cDNAsp: | |
| 5′-AGC TAA GCT TCA CAA GAC AAG GCA | (SEQ ID NO 9) |
| ACC AGA TTT TTT CTT CC-3′, | |
| RhoB-cDNAst: | |
| 5′-ATC AAG ATC TAT GGC GGC CAT CCG | (SEQ ID NO 10) |
| CAA GAA GCT GGTG-3′, | |
| RhoB-cDNAsp: | |
| 5′-AGC TAA GCT TAT CAT AGC ACC TTG | (SEQ ID NO 11) |
| CAG CAG TTG ATG CAG CC-3,′ | |

pCMV-β-gal is purchased from Promega. pcDNA-HDAC1 expression vectors is made according to conventional methods, and pcDNA-HDAC6 expression vector which can express functional HDAC6 in transfected cells was a generous gift from Dr. E. Verdin's lab (UCSF) (Fischle, W. et al., Mol Cell 9, 45-57 (2002) and Chen, L et al., Science 293, 1653-7 (2001)). All constructs are confirmed by sequencing.
Apoptosis Assay.

HeLa and HCT116 cells are transfected with RhoA or RhoB expression plasmids (described in methods) or with the pcDNA3.1 empty vector. The cells are harvested 48 h after transfection by trypsinization, washed with PBS, and fixed in 70% ethanol. The cells are stained in PBS/phosphate-citric acid buffer (40 mM Na₂HPO₄, 20 mM citric acid, pH 7.8) containing 10% FBS, 100 μg/ml RNase A and 50 μg/ml propidium iodide. Flow cytometry is performed using a MoFlo (Cytomation, Fla.). The proportion of cells exhibiting sub-G1 phase DNA is scored as apoptotic cells. The percentage of cells undergoing apoptosis in the cell population is calculated by Muticycle software (Phoenix Flow System).

Luciferase and β-gal Activity Assays.

For the reporter activity assay, transfected cells are lysed in 1× reporter lysis buffer (Promega), 48 h after transfection. As a control, pGL3 basic vector (Promega), a promoterless luciferase vector, is also transfected. Luciferase and β-gal activity assays are performed according to the manufacturer's instructions (Promega). Luciferase activity is measured using a microtiter plate luminometer (Dynex Technologies, Chantilly, Va.).

Chromatin Immunoprecipitation (CHIP).

Chromatin immunoprecipitation (CHIP) analysis is conducted as described previously in Boyd, K. E. et al., PNAS USA 95 13887-92 (1998) and Moreno, C. S. et al., Immunity 10, 143-51 (1999). Briefly, formaldehyde is added directly to H1299 cell culture media to a final concentration of 1%. Fixed cells are washed and resuspended in cell lysis buffer (5 mM PIPES, pH 8.0, 85 mM KCl, 0.5% NP-40, and a cocktail of protease inhibitors), and the nuclei are spun down and resuspended in nuclear lysis buffer (50 mM Tris-HCl, pH 8.1, 10 mM EDTA, 1% SDS). Chromatin is isolated and fragmented by sonication to an average size of less than 1 kb. The chromatin is immunoprecipitated either with anti-HDAC1, anti-HDAC2 and anti-HDAC6 antibodies, or without antibody (Upstate Biotechnology and Santa Cruz Biotechnology). Immune complexes are washed stringently, DNA is eluted, and crosslinking is reversed as described in Boyd and Moreno. DNA is purified and used as the PCR template. The supernatant fraction from the reaction lacking primary antibody is saved as the “chromatin input”. 5 μl of DNA (20% of the immuno-precipitated chromatin preparation) is used to amplify the RhoB promoter region from −557 to −412 relative to the translation initiation site, PCR products are separated on a 1.5% agarose gel, stained with ethidium bromide, and the image is recorded.

Cell Transfection with Antisense Oligonucleotides.

H1299 cells are transfected with HDAC1 antisense oligonucleotides NAS 6887, HDAC1 mismatch oligonucleotide NAS 7618, luciferase antisense oligonucleotide NAS 5596, or mock transfected. The following are the sequences of the modified oligonucleotides:

| | |
|---|---|
| NAS 6887- | |
| gct gtAs CsTsCs CsGsAs Csat gtt | (SEQ ID NO 12) |
| NAS 7618- | |
| gct ttAs CsGsCs CsTsAs Csag gtt | (SEQ ID NO 13) |
| NAS 5596- | |
| cct taCs CsTsGs CsTsAs Gsct ggc | (SEQ ID NO 14) |

modified such that “s” stands for phosphorothioate, upper case refers to 2′-H, lower case refers to 2′-O-methoxyethyl and “c” refers to 2′-methoxyethyl 5-methylcytidine. Transfection is performed using LIPOFECTIN (Life Technologies) as a carrier according to the manufacturer's instruction. The transfection mixture is prepared in OPTI-MEM (Life Technologies) containing 6 μl LIPOFECTIN per 400 nM oligonucleotide. Transfection is carried out for 4 h, and is repeated twice on two consecutive days. Cells are harvested 72 h after the first transfection. RNA and proteins are isolated using Trizol reagent (Life Technologies) according to the manufacturer's instruction. Traces of DNA are removed from RNA samples using DNA-free (Ambion, Austin, Tex.) according to the manufacturer's protocol. RNA concentration is measured on CytoFluor Multi-well plate reader (PerSeptive Biosystems, Framingham, Mass.) using RIBO GREEN RNA Quantification kit (Molecular Probes, Eugene, Oreg.).
Real Time RT-PCR

Real time RT-PCR is performed using qPCR Core Kit (Eurogentec, Belgium) in an ABI PRISM 7700 Sequence Detector (Applied Biosystems, Forster City, Calif.) following the manufacturer's instructions.
HDAC1 forward primer (GCGATGAGGACGAAGACGAC) (SEQ ID NO 15),
HDAC1 reverse primer (TCACAGGCAATTCGTTTGTCA) (SEQ ID NO 16) and
HDAC1 TaqMan probe (CTGACAAGCGCATCTCGATCTGCTCC) (SEQ ID NO 17) containing 5′ FAM fluorescent reporter and 3′ TAMRA quencher dyes, are used in the reaction at the final concentrations of 200 nM for primers and 100 nM for fluorescent probe. RNase inhibitor (Applied Biosystems) and MuLV Reverse Transcriptase (Applied Biosystems) are used at the final concentration of 0.2 U/μl and 0.125 U/μl respectively. Relative measurement of the amplified product is performed using the comparative CT method as described in the manufacturer's manual (Applied Biosystems, ABI Prism 7700 sequence detection system, user Bulletin #2).

### Example 10

### RhoB is Differentially Expressed in Transformed Cell Lines

DNA microarray analysis was used to characterise RhoB, RhoA, and RhoC expression in cancer cell lines including those of liver, kidney, colon, breast, brain and blood origin (Affymetrix chip HG U95Av2). RhoB was found to have very low expression levels in these cancer cell lines, compared with the expression levels of RhoA and RhoC.

### Example 11

### Overexpression of RhoB Induces Apoptosis in HeLa and HCT116 Cells

In order to further analyse the role of RhoB in tumour cells, the effect of Rho proteins overexpression on apoptosis was investigated. RhoB and RhoA full-length cDNAs were cloned into mammalian expression vectors (pcDNA3.1, Invitrogen, Carlsbad, Calif.). pcDNA-RhoB, pcDNA-RhoA or pcDNA3 were then transfected into HeLa and HCT116 cells and apoptosis assays performed. Cells exhibiting sub-G1-phase DNA were scored as apoptotic cells, and the percentage of cells undergoing apoptosis in the cell population was calculated. Results indicate that overexpression of RhoB in these cells led to an approximate 3-fold increase in the number of apoptotic cells relative to the control. In contrast, overexpression of RhoA in these cells had no obvious effect on apoptosis.

### Example 12

### The Human and Mouse RhoB Promoters

To understand the induction of RhoB by TPX, and to determine whether this induction could be mapped to the RhoB promoter, the region of the human RhoB gene (GenBank accession number: AC023137) from −1224 to −253 (relative to the translation initiation site, +1 ATG) (see Table 6) was cloned upstream of a luciferase reporter gene. Based on EST and transcripts data analysis, a transcription initiation site was predicted at position-428 G. Comparison of the sequences of human and mouse RhoB 5′ upstream regions (mouse: EMBL accession number Y09248; human: GenBank accession number: AC023137), revealed that the two promoters are highly conserved around the potential transcription initiation site. Furthermore, these promoters also share many of the same putative transcription factors binding sites.

TABLE 6
Sequence of the 5′-noncoding region of human Rho B
(SEQ ID NO. 18)

−1224
CGGGATCAGAGTTCATAGTGAAAAGAGGGGCCGAAGGGGCTTCTTCCTGT
−1174
CCGGTAACTTCCTCCAAGTGTTCGAGCCTCGACCTTCCCTTTCCGTTGTC
−1124
AAGCTTGGGCCACTCCGCACCTCTTCCCTTAATCTTCACCTAGAGACCTA
−1074
AGCTGGGGGTTGGGAAGGGTAGGGGCGGCGGGACTTGGAAGAGCCAGTTT
−1024
GCAGCCAGCCGGTCGCCTCTCGGGTCCAAACCCGAGGCTGGCCCACGGCG
−974
AGTACCCGGGTGGGGCCCTAAACCACAGGAGGGGCCACCTCCAAAAGAAA
−924
AGAGAAAACACTCTTGTTTGGGAACAAAAGTGTGTGTGTGGGGTGTGTGT
−874
GTGTGTGTGTGTGTGTGTGTGTGTCTGTTTATTTAAAAACAAAACCACGT
−824
TAAAAGAGCTGCCCCTCCCCCACAGGCCCGACCACCCGCCGGGAGTTTGC
−774
CAGGAAGAGGGGCAATTCTGAATGGGAGTCGCCAACGCCCCACTGAGTGA
−724
AGCCTGTCTCGGAACCGCTCGCCCAGACCCTGGAGGCTCCAGACAGCCAG
−674
CTCCCGGACCCCGCGCGCAAACGCTGCGGCGAAGGAGGGGACCCGGGTAC
−624
CGCCAGAGCCCCGCAGCGGCAGCAGCAGCGCAGACTCCCCGGTCGATGCC
−574
TCTCCCAGCCCGGCGGCCTGGGCCGTCAATCAAGCTGGCCCTGCCCCGCC
−524
CTCGAGCTGCAGGGGGCGGCCAATCAGAGATAAGCTCCGCAGCGATGAGC
−474
TCAGCCGGCTGGTTTCCCATTGGACGGCTATATTAAGAAAGTGGCCGGAC
−424
TCTTTAAATAGCGGGCGCTAGGGCCGCAGCCCTCATCTGCCACCGCAGTC
−374
TGGTTGGAGCTGTTGTCTTGTATGCTCAGCGAGGCCCGGAGAGACCCGGG
−324
AGAGAGCTAGGCCGAGTCCACCGCCCGAGTCTGCTGCCCGAGCCCGCGTT
−274
ACGCACAAAGCCGCCGATCCCCGGCCTGGGGTGAGCAGAGCGACCACCGC
−224
CCGGGAGCAGCGCGGCGAGACGCACGGTGCGCCCTATGCCCCCGCGCCCC
−174
CACCGCCCCCGCCGCGGCAGCCGAAGCGCAGCGAGAGAACGCGCCACCGC
−124
GGGGCCCGGGTGCAGCTAGCGACCCTCTCGCCACCTGCGCGCAGCCCGAG
−74
GTGAGCAGTGAGCGGCGAGCGGGAGGGCAGCGAGGCGTTCGCGGGCCCCC
−24
TCCTGCTGCCCGGGCCCGGCCCTCATG

Bold G at position-428 is a putative transcription start site, and bold ATTGG at positions-456-452 is a putative Sp1 site.

RhoB Promoter Activity is Induced by TPX and an Inverted CCAAT box is Crucial for this Induction

To determine whether the RhoB promoter was induced by TPX, 0.97 kb (−1224 to −253) of RhoB 5′-upstream genomic region was cloned into a luciferase reporter vector, and a series of 5′ deletion constructs were prepared. The effect of TPX on the transcriptional activity of the RhoB promoter was determined in H1299 cells transfected with these constructs after TPX treatment. Results indicate that luciferase activity driven by the RhoB promoter was induced to about 10-fold following TPX treatment. To define the region required for the TPX responsiveness, a series of RhoB promoter 5′ deletion constructs were transfected into H1299 cells. All the constructs generated were induced by TPX to a similar extent as shown by the luciferase activities.

Examination of the RhoB promoter sequence between −497 and −392, which was also induced by TPX, revealed the presence of two potential transcription factor binding sites, a putative TATA box at position-438 and an inverted CCAAT box (5′-ATTGG-3′) at position-451 with a five nucleotide space between these two boxes. To define which, if any, of these boxes were required for TPX induction, the activation of luciferase constructs containing point mutations in the CCAAT and TATA boxes, as well as a construct containing an insertion of three nucleotides that changed the distance between these two boxes, were examined. H1299 cells transfected with these constructs were treated with 30 nM TPX or 0.1% DMSO for 24 h, and luciferase activity was measured in cell lysates. Data were normalized to luciferase activity from DMSO-treated cells transfected with the same constructs. Three independent experiments were performed. Results indicate that the response of the RhoB promoter to TPX was dramatically reduced to the promoterless vector control level when the inverted CCAAT box (5′-ATTGG-3′) was mutated to 5′-TTTAG-3′. However, mutation of the TATA box (5′-TATATTAA-3′) to 5′CAGATCAA-3′ had no significant effect on TPX induction. In addition, the insertion of three nucleotides (AAA) between the CCAAT and TATA boxes had no effect. These data indicate that the inverted CCAAT box is pivotal for the induction of the RhoB promoter by TPX.

RhoB Promoter is Associated with and Inhibited by HDAC1.

To determine whether the induction of the RhoB promoter by TPX is related to in vivo recruitment of HDAC complexes to the promoter, the association of HDACs with the RhoB promoter was investigated by chromatin immunoprecipition (CHIP) assay. H1299 cells were fixed in 1% formaldehyde, and chromatin lysates were immunoprecipited with specific antibodies against HDAC1, 2 and 6. Results indicate that the RhoB promoter fragment −557 to −412 was found to be associated with HDAC1 and HDAC2, but was not associated with HDAC6. As a control, chromatin was immunoprecipitated in the absence of specific antibodies, and in these samples, the RhoB promoter fragment was not detected.

As shown earlier, the change in the induction of luciferase activity by TPX was found to be mediated only by the inverted CCAAT box in the RhoB promoter. To investigate the direct effect of HDACs on the RhoB promoter, expression constructs of HDAC1, HDAC6 (pcDNA-HDAC1 and pcDNA-HDAC6) and empty vector were co-transfected with pGL3RhoB5-WT and pGL3RhoB-MT-CAAT reporter constructs in H1299 cells. The cells were harvested at 48 h after transfection and luciferase activity was determined. The activity of vector control was set at 100%, and luciferase activity was normalized to β-gal activity. Results indicate that the activity of the RhoB promoter is repressed by overexpression of HDAC1, but not by HDAC6, revealing that these two HDACs have different effects on RhoB promoter activity. However, HDAC1 overexpression has no effect on the construct containing the mutation in the inverted CCAAT box. These results demonstrate that RhoB promoter activity is regulated by HDAC1 but not by HDAC6.

### Example 13

### Expression of Rhob is Induced by Blocking of HDAC1 Synthesis

To determine the effect of specific HDAC1 blockade on RhoB expression, antisense oligonucleotides against HDAC1 were used. Potent antisense oligonucleotides against HDAC1 as well as oligonucleotides with mismatched nucleotides were synthesized and transfected into H1299 cells as specifically disclosed herein and as described in Examples 6-9. One antisense oligonucleotides in particular, NAS 6887, was found to have very potent inhibition on HDAC1 mRNA level and was subsequently used. RNA and protein were isolated from H1299 cells transfected with the HDAC1 antisense oligonucleotides (NAS 6887), the corresponding mismatch (NAS 7618) and the unrelated luciferase control oligonucleotide (NAS 5596), and were analysed by real time RT-PCR and Western blot for HDAC1 and RhoB expression level. Results indicate that the expression of HDAC1 was efficiently blocked by NAS 6887 and RhoB protein level was significantly induced. In contrast, no effects on RhoB expression were found in cells transfected with the NAS 7618 and NAS 5596 oligonucleotides.

What is claimed is:

1. An antisense compound 8 to 30 nucleotides in length targeted to a 5″UTR, a coding region or a 3″ UTR of a nucleic acid encoding a trapoxin regulated gene selected from the group consisting of those disclosed in Table 5 and wherein said antisense compound inhibits the expression of said trapoxin regulated gene.

2. The antisense compound of claim 1 which is an antisense oligonucleotide.

3. The antisense compound of claim 2 wherein the antisense oligonucleotide comprises at least one modified internucleoside linkage.

4. The antisense compound of claim 3 wherein the modified internucleoside linkage is a phosphororthioate linkage.

5. The antisense compound of claim 2 wherein the antisense oligonucleotide comprises at least one modified sugar moiety.

6. The antisense compound of claim 5 wherein the modified sugar moiety is a 2′-O -methoxyethyl sugar moiety.

7. The antisense compound of claim 2 wherein the antisense oligonucleotide comprises at least one modified base.

8. The antisense compound of claim 2 which is a chimeric oligonucleotide.

9. A method for treating a proliferative disease in a subject in need thereof, comprising providing a therapeutically effective amount of a composition comprising an inducer of RhoB to said subject.

10. The method of claim 9 wherein said proliferative disease is cancer.

11. A pharmaceutical composition comprising one or more inhibitors of any one or more trapoxin down-regulated genes selected from the group consisting of those disclosed in Table 5.

12. A pharmaceutical composition comprising one or more an inducers of any onre or more trapoxin up regulated genes selected from the group consisting of those disclosed in Table 5.

13. A method for screening a compound for HDAC inhibitory activity, comprising administering said compound to a subject and assaying for RhoB mRNA levels in a biological sample from said subject wherein increased levels compared to controls indicate a compound possessing HDAC inhibitory activity.

14. A method for screening a compound for HDAC inhibitory activity, comprising administering said compound to a subject and assaying for RhoB protein levels in a biological sample from said subject wherein increased levels compared to controls indicate a compound possessing HDAC inhibitory activity.

15. A method for screening a compound for HDAC inhibitory activity, comprising administering said compound to an in vitro cellular screening system and assaying for RhoB mRNA levels in said system wherein increased levels compared to controls indicate a compound possessing HDAC inhibitory activity.

16. A method for screening a compound for HDAC inhibitory activity, comprising administering said compound to an in vitro cellular screening system and assaying for RhoB protein levels in said system wherein increased levels compared to controls indicate a compound possessing HDAC inhibitory activity.

17. A method for inhibiting HDAC activity in a subject, comprising administering to said subject a substance having the ability to upregulate RhoB, in an amount sufficient to inhibit HDAC activity in said subject.

18. A method for treating conditions associated with abnormal HDAC activity in a subject in need thereof, comprising administering to said subject a therapeutically effective amount of a substance having the ability to upregulate levels of Rho B in said subject.

19. A method for monitoring the progress of treatment of a disease associated with abnormal HDAC activity in a subject comprising monitoring message levels of one or more trapoxin-regulated genes selected from the group consisting of those disclosed in Table 5 in a sample from said subject.

20. A method for monitoring the progress of treatment of a disease associated with abnormal HDAC activity in a subject comprising monitoring protein levels of one or more trapoxin regulated genes selected from the group consisting of those disclosed in Table 5 in a sample from said subject.

21. The method of claim 19 wherein said trapoxin regulated gene is RhoB.

22. The method of claim 20 wherein said trapoxin regulated gene is RhoB.

23. A pharmaceutical composition comprising the antisense oligonucleotide of claim 1.

24. A method of modulating expression of a trapoxin regulated gene selected from the group consisting of those disclosed in Table 5 in cells or tissues comprising contacting said cells or tissues with one or more antisense oligonucleotides to said trapoxin regulated gene.

25. A method of treating, preventing or ameliorating a condition associated with abnormal expression of a trapoxin down-regulated gene selected from the group consisting of those disclosed in Table 5 in a subject comprising administering a therapeutically effective amount of one or more antisense oligonucleotides directed to said trapoxin down-regulated gene.


SEQUENCE LISTING (18 sequences)

SEQ ID NO: 1 - DNA, 78 bp, Artificial Sequence
  primer
aaacgacggcacacttcgaaattaatacgactcactatagggaggcggtttttttttttttttttttttttttttttt

SEQ ID NO: 2 - DNA, 22 bp, Artificial Sequence
  primer
gtccttttgacactgctctaac

SEQ ID NO: 3 - DNA, 22 bp, Artificial Sequence
  primer
cttgagatgacactgctctaac

SEQ ID NO: 4 - DNA, 22 bp, Artificial Sequence
  primer
tcagatgttcgcccttcaccag

SEQ ID NO: 5 - DNA, 24 bp, Artificial Sequence
  primer
gttacagcgtacaagtgtggtcag

SEQ ID NO: 6 - DNA, 23 bp, Artificial Sequence
  primer
tcacaggggtacagaaattatcc

SEQ ID NO: 7 - DNA, 23 bp, Artificial Sequence
  primer
gaccaaatgcagtgagagacaag

SEQ ID NO: 8 - DNA, 37 bp, Artificial Sequence
  primer
atcaagatctatggctgccatccggaagaaactggtg

SEQ ID NO: 9 - DNA, 41 bp, Artificial Sequence
  primer
agctaagcttcacaagacaaggcaaccagattttttcttcc

SEQ ID NO: 10 - DNA, 37 bp, Artificial Sequence
  primer
atcaagatctatggcggccatccgcaagaagctggtg

SEQ ID NO: 11 - DNA, 41 bp, Artificial Sequence
  primer
agctaagcttatcatagcaccttgcagcagttgatgcagcc

SEQ ID NO: 12 - DNA, 18 bp, Artificial Sequence
  antisense oligonucleotide
gctgtactccgacatgtt

SEQ ID NO: 13 - DNA, 18 bp, Artificial Sequence
  antisense oligonucleotide
gctttacgcctacaggtt

SEQ ID NO: 14 - DNA, 18 bp, Artificial Sequence
  antisense oligonucleotide
ccttacctgctagctggc

SEQ ID NO: 15 - DNA, 20 bp, Artificial Sequence
  primer
gcgatgaggacgaagacgac

SEQ ID NO: 16 - DNA, 21 bp, Artificial Sequence
  primer
tcacaggcaattcgtttgtca

SEQ ID NO: 17 - DNA, 26 bp, Artificial Sequence
  primer
ctgacaagcgcatctcgatctgctcc

SEQ ID NO: 18 - DNA, 1227 bp, Homo sapiens
cgggatcagagttcatagtgaaaagaggggccgaaggggcttcttcctgtccggtaacttcctccaagtgttcgagcctcgaccttccctttccgttgtcaagcttgggccactccgcacctcttcccttaatcttcacctagagacctaagctgggggttgggaagggtaggggcggcgggacttggaagagccagtttgcagccagccggtcgcctctcgggtccaaacccgaggctggcccacggcgagtacccgggtggggccctaaaccacaggaggggccacctccaaaagaaaagagaaaacactcttgtttgggaacaaaagtgtgtgtgtggggtgtgtgtgtgtgtgtgtgtgtgtgtgtgtgtctgtttatttaaaaacaaaaccacgttaaaagagctgcccctcccccacaggcccgaccacccgccgggagtttgccaggaagaggggcaattctgaatgggagtcgccaacgccccactgagtgaagcctgtctcggaaccgctcgcccagaccctggaggctccagacagccagctcccggaccccgcgcgcaaacgctgcggcgaaggaggggacccgggtaccgccagagccccgcagcggcagcagcagcgcagactccccggtcgatgcctctcccagcccggcggcctgggccgtcaatcaagctggccctgccccgccctcgagctgcagggggcggccaatcagagataagctccgcagcgatgagctcagccggctggtttcccattggacggctatattaagaaagtggccggactctttaaatagcgggcgctagggccgcagccctcatctgccaccgcagtctggttggagctgttgtcttgtatgctcagcgaggcccggagagacccgggagagagctaggccgagtccaccgcccgagtctgctgcccgagcccgcgttacgcacaaagccgccgatccccggcctggggtgagcagagcgaccaccgcccgggagcagcgcggcgagacgcacggtgcgccctatgcccccgcgcccccaccgcccccgccgcggcagccgaagcgcagcgagagaacgcgccaccgcggggcccgggtgcagctagcgaccctctcgccacctgcgcgcagcccgaggtgagcagtgagcggcgagcgggagggcagcgaggcgttcgcgggccccctcctgctgcccgggcccggccctcatg